(19) United States
(12) Patent Application Publication
(10) Pub. No.: US 2026/0315014 A1
(43) Pub. Date: Oct. 15, 2026

(54) MEDICAL IMAGING METHODS AND SYSTEMS

(71) Applicant: SHANGHAI UNITED IMAGING HEALTHCARE CO., LTD., Shanghai (CN)

(72) Inventors: Jia CHEN, Shanghai (CN); Jie YANG, Shanghai (CN); Ruijie SHI, Shanghai (CN); Hao ZHANG, Shanghai (CN)

(73) Assignee: SHANGHAI UNITED IMAGING HEALTHCARE CO., LTD., Shanghai (CN)

(21) Appl. No.: 19/690,114

(22) Filed: May 28, 2026

Related U.S. Application Data
(63) Continuation of application No. PCT/CN2024/143319, filed on Dec. 27, 2024.

(30) Foreign Application Priority Data
Dec. 27, 2023 (CN) 202311828468.5
Dec. 27, 2023 (CN) 202311836412.4
Dec. 29, 2023 (CN) 202311872469.X

(51) Int. Cl. A61B 6/04; G06T 7/11; G06T 7/20; G06T 7/73

(52) U.S. Cl. CPC A61B 6/0492; G06T 7/11; G06T 7/20; G06T 7/73

(57) ABSTRACT

The embodiments of the present disclosure provide medical imaging methods and systems. A method, implemented by a computing device including at least one processor and at least one storage device, comprises: obtaining a first target image of a target subject, the first target image being taken by a camera system; determining a marker in the first target image, the marker being configured to characterize a scanning position; and determining a scanning range of a medical imaging device for scanning the target subject based on the marker.

### CROSS-REFERENCES TO RELATED APPLICATIONS

This application is a continuation of International Application No. PCT/CN2024/143319, filed on Dec. 27, 2024, which claims priority to Chinese Patent Application No. 202311836412.4, filed on Dec. 27, 2023, Chinese Patent Application No. 202311828468.5, filed on Dec. 27, 2023, and Chinese Patent Application No. 202311872469.X, filed on Dec. 29, 2023, the entire contents of each of which are incorporated herein by reference.

### TECHNICAL FIELD

The present disclosure relates to the field of medical imaging technology, and in particular to a medical imaging method and system.

### BACKGROUND

Medical imaging is one of the main examination methods in the medical field. When using medical imaging technologies such as computed tomography (CT), magnetic resonance imaging (MRI), positron emission tomography (PET) to scan a patient, it is necessary to determine a scanning range first. However, due to limitations such as distance and viewing angle, the scanning range is difficult to determine, resulting in large errors in the scanning result, and the patient may also be exposed to unnecessary radiation.

Therefore, it is desirable to provide a medical imaging method and system to improve the accuracy of determining the scanning range.

### SUMMARY

One or more embodiments of the present disclosure provide a method, implemented by a computing device including at least one processor and at least one storage device. The method may comprise obtaining a first target image of a target subject, the first target image being taken by a camera system; determining a marker in the first target image, the marker being configured to characterize a scanning position; and determining a scanning range of a medical imaging device for scanning the target subject based on the marker.

In some embodiments, the marker may include at least one of an imaging position, a center line of the target subject, or a region of interest (ROI) of the target subject. The imaging position may be configured to characterize a position on the target subject capable of being scanned by the medical imaging device.

In some embodiments, the determining a marker in the first target image may include: determining the imaging position according to a scanning protocol and bed position information of a scanning bed.

In some embodiments, the determining a marker in the first target image may include: identifying the target subject in the first target image; and determining the center line of the target subject according to the identified target subject in the first target image.

In some embodiments, the determining a marker in the first target image may include: identifying the target subject in the first target image; acquiring scanning information of the target subject, the scanning information indicating the ROI of the target subject; and marking, based on the scanning information and the identified target subject in the first target image, a contour of the ROI in the first target image.

In some embodiments, the marking, based on the scanning information and the identified target subject in the first target image, a contour of the ROI in the first target image may include: marking the contour of the ROI in the first target image using a recognition model, an input of the recognition model including the first target image and the scanning information.

In some embodiments, the input of the recognition model may further include at least one of a medical history of the target subject or a historical medical image of the ROI of the target subject.

In some embodiments, the determining a scanning range of a medical imaging device for scanning the target subject based on the marker may include: displaying the first target image including the marker; and determining, based on the displayed marker, the scanning range of the target subject.

In some embodiments, the first target image may include real-time images of the target subject during a movement process of a scanning bed of the medical imaging device, and the real-time images may reflect a real-time relative position relationship between the target subject and the imaging position during the movement process of the scanning bed; and determining, based on the displayed marker, the scanning range of the target subject may include: during the movement process of the scanning bed, determining the scanning range of the target subject according to the real-time relative position relationship.

In some embodiments, the during the movement process of the scanning bed, determining the scanning range of the target subject according to the real-time relative position relationship may include: during the movement process of the scanning bed, receiving a confirmation instruction, the confirmation instruction including a confirmation instruction of a start position and a confirmation instruction of an end position; and determining the scanning range of the target subject according to the start position and the end position, the start position being a first target position related to the scanning bed, the end position being a second target position related to the scanning bed.

In some embodiments, the determining, based on the displayed marker, the scanning range of the target subject may include: determining at least one image position in the first target image by receiving a user instruction performed in the displayed image; and determining the scanning range of the target subject based on the at least one image position.

In some embodiments, the determining a scanning range of a medical imaging device for scanning the target subject based on the marker may include: automatically determining at least one image position in the first target image based on the ROI of the target subject; and determining the scanning range of the target subject based on the at least one image position.

In some embodiments, the camera system may include at least one of a first camera device and a second camera device mounted inside a scanning cavity of the medical imaging device. The first camera device may be disposed at a top of the scanning cavity. The second camera device may be disposed on a side of the scanning cavity.

In some embodiments, the first target image of the target subject may include a first target image of a top view of the target subject taken by the first camera device.

In some embodiments, the first target image of the target subject may include a second image of a side view of the target subject taken by the second camera device.

In some embodiments, the method may further comprise: controlling a movement process of a scanning bed of the medical imaging device; and in response to determining that the scanning range matches the imaging position, controlling the medical imaging device to scan the target subject.

In some embodiments, the method may further comprise acquiring scanning data of the scanning range of the target subject; during acquiring the scanning data, obtaining at least two second target images of the target subject generated based on the camera system, the at least two second target images corresponding to different time points; determining motion change information of the target subject according to the at least two second target images; and generating a corrected scanning image by performing, based on the motion change information, artifact correction on the scanning data.

In some embodiments, the method may further comprise obtaining a target depth image taken by the camera system and a historical depth image captured in the same scene as the target depth image by the camera system prior to the target depth image; determining an operation status of the camera system according to at least one of the target depth image and the historical depth image, the operation status including at least one of a fault status, an abnormal tendency status, or a normal status; and sending prompt information to a user based on the operation status of the camera system.

In some embodiments, a position of the scanning range may be the same or substantially the same as that of an ROI of the target subject.

One or more embodiments of the present disclosure provide a method, implemented by a computing device including at least one processor and at least one storage device. The method may comprise obtaining a target depth image taken by a camera system and a historical depth image captured in the same scene as the target depth image by the camera system prior to the target depth image, the camera system being mounted in a medical device operation room; determining an operation status of the camera system according to at least one of the target depth image or the historical depth image, and the operation status including a fault status, an abnormal tendency status, or a normal status; and sending prompt information to a user based on the operation status of the camera system.

One or more embodiments of the present disclosure provide a system. The system may comprise at least one storage medium including a set of instructions; at least one processor in communication with the at least one storage medium. When executing the set of instructions, the at least one processor may be directed to cause the system to perform operations comprising: obtaining a first target image of a target subject, the first target image being taken by a camera system; determining a marker in the first target image, the marker being configured to characterize a scanning position; and determining a scanning range of a medical imaging device for scanning the target subject based on the marker.

One or more embodiments of the present disclosure provide a system. The system may comprise an image acquisition module configured to obtain a first target image of a target subject, the first target image being taken by a camera system; a marker determination module configured to determine a marker in the first target image, the marker being configured to characterize a scanning position; and a range determination module configured to determine a scanning range of a medical imaging device for scanning the target subject based on the marker.

One or more embodiments of the present disclosure provide a non-transitory computer readable medium, comprising executable instructions that, when executed by at least one processor, may direct the at least one processor to perform a method, the method comprising: obtaining a first target image of a target subject, the first target image being taken by a camera system; determining a marker in the first target image, the marker being configured to characterize a scanning position; and determining a scanning range of a medical imaging device for scanning the target subject based on the marker.

One or more embodiments of the present disclosure provide a system. The system may comprise at least one storage medium including a set of instructions; at least one processor in communication with the at least one storage medium. When executing the set of instructions, the at least one processor may bee obtaining a target depth image taken by a camera system and a historical depth image captured in the same scene as the target depth image by the camera system prior to the target depth image, the camera system being mounted in a medical device operation room; determining an operation status of the camera system according to at least one of the target depth image or the historical depth image, and the operation status including a fault status, an abnormal tendency status, or a normal status; and sending prompt information to a user based on the operation status of the camera system.

One or more embodiments of the present disclosure provide a system. The system may comprise an image acquisition module configured to obtain a target depth image taken by a camera system and a historical depth image captured in the same scene as the target depth image by the camera system prior to the target depth image, the camera system being mounted in a medical device operation room; a status determination module configured to determine an operation status of the camera system according to at least one of the target depth image or the historical depth image, and the operation status including a fault status, an abnormal tendency status, or a normal status; and an information prompt module configured to send prompt information to a user based on the operation status of the camera system.

One or more embodiments of the present disclosure provide a non-transitory computer readable medium, comprising executable instructions that, when executed by at least one processor, may direct the at least one processor to perform a method, the method comprising: obtaining a target depth image taken by a camera system and a historical depth image captured in the same scene as the target depth image by the camera system prior to the target depth image, the camera system being mounted in a medical device operation room; determining an operation status of the camera system according to at least one of the target depth image or the historical depth image, and the operation status including a fault status, an abnormal tendency status, or a normal status; and sending prompt information to a user based on the operation status of the camera system.

One or more embodiments of the present disclosure provide a system. The system may comprise at least one storage medium including a set of instructions; at least one processor in communication with the at least one storage medium. When executing the set of instructions, the at least one processor may be directed to cause the system to perform operations comprising: obtaining scanning data of a target subject acquired by scanning the target subject by a medical imaging device; during acquiring the scanning data, obtaining at least two second images of the target subject generated based on a camera system, the at least two second target images corresponding to different time points; determining motion change information of the target subject according to the at least two second images; and generating a corrected scanning image by performing, based on the motion change information, artifact correction on the scanning data.

One or more embodiments of the present disclosure provide a system. The system may comprise a data acquisition module configured to obtain scanning data of a target subject acquired by scanning the target subject by a medical imaging device; an image acquisition module configured to obtain, during acquiring the scanning data, at least two second images of the target subject generated based on a camera system, the at least two second target images corresponding to different time points; a motion determination module configured to determine motion change information of the target subject according to the at least two second images; and a data correction module configured to generate a corrected scanning image by performing, based on the motion change information, artifact correction on the scanning data.

One or more embodiments of the present disclosure provide a non-transitory computer readable medium, comprising executable instructions that, when executed by at least one processor, may direct the at least one processor to perform a method, the method comprising: obtaining scanning data of a target subject acquired by scanning the target subject by a medical imaging device; during acquiring the scanning data, obtaining at least two second images of the target subject generated based on a camera system, the at least two second target images corresponding to different time points; determining motion change information of the target subject according to the at least two second images; and generating a corrected scanning image by performing, based on the motion change information, artifact correction on the scanning data.

Additional features may be set forth in part in the description which follows, and in part may become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical imaging system according to some embodiments of the present disclosure;

FIG. 2 is a schematic diagram illustrating an exemplary medical imaging system according to some embodiments of the present disclosure;

FIG. 3 is a flowchart illustrating an exemplary medical imaging method according to some embodiments of the present disclosure;

FIG. 4 is a schematic diagram illustrating an exemplary medical imaging system in operation according to some embodiments of the present disclosure;

FIG. 5 is a schematic diagram illustrating a display interface of an exemplary medical imaging system according to some embodiments of the present disclosure;

FIG. 6 is a schematic diagram illustrating another display interface of an exemplary medical imaging system according to some embodiments of the present disclosure;

FIG. 7 is a schematic diagram illustrating an exemplary medical imaging method according to some embodiments of the present disclosure;

FIG. 8 is a schematic diagram illustrating an exemplary medical imaging method according to some embodiments of the present disclosure;

FIG. 9 is a schematic diagram illustrating an exemplary medical imaging method according to some embodiments of the present disclosure;

FIG. 10 is a schematic diagram illustrating an exemplary medical imaging method according to some embodiments of the present disclosure;

FIG. 11 is a flowchart illustrating an exemplary camera detection method according to some embodiments of the present disclosure;

FIG. 12 is a flowchart illustrating an exemplary camera detection method according to some embodiments of the present disclosure;

FIG. 13 is a flowchart illustrating an exemplary medical imaging method according to some embodiments of the present disclosure; and

FIG. 14A and FIG. 14B are schematic diagrams illustrating a mounting position of a camera system of an exemplary medical imaging system according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but is to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the terms “system,” “engine,” “unit,” “module,” and/or “block” used herein are one method to distinguish different components, elements, parts, sections, or assemblies of different levels in ascending order. However, the terms may be displaced by other expressions if they may achieve the same purpose.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present unless the context clearly indicates otherwise. As used herein, the term “and/or” includes any or all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

In medical imaging examinations, a scanning range needs to be determined first. Taking a CT system as an example, since a scanned subject is located in an aperture for scanning, it is difficult for a user (e.g., a doctor, etc.) to accurately grasp the scanning range due to distance and view angle limitations. The commonly used strategy is to first perform a scout scan to obtain a scout image to preliminarily locate a region of interest (ROI) of the scanned subject, and then determine a scanning range according to the scout image. This method requires the scanned subject to constantly adjust his or her posture to obtain a better viewing angle, which may cause large errors, and the patient may receive an unnecessary radiation dose as an exposure range of the patient cannot be effectively estimated before imaging. In addition, in order to determine the scanning range, the user often locates the sagittal and coronal planes of the patient based on an external laser light, and then moves the scanning part (ROI) of the patient to an internal laser position in the aperture. However, the actual scanning range in the aperture of the CT system is difficult to visually observe, thus the positioning is difficult, and the laser light has low positioning accuracy and takes a long time.

The embodiments of the present disclosure provide a medical imaging method and system. The medical imaging method may include obtaining a first target image of a target subject, the first target image being taken by a camera system. The medical imaging method may include determining a marker in the first target image, the marker being configured to characterize a scanning position. The medical imaging method may further include determining a scanning range of a medical imaging device for scanning the target subject based on the marker.

According to the embodiments of the present disclosure, the scanning position is located by using the marker on the image of the target subject, and then the scanning range is determined before the actual scanning, thereby improving the accuracy of the determined scanning range and improving the scanning quality. Meanwhile, the scout image based on radioactive scanning is avoided (the scanning range of the scanned subject is determined without pre-scanning the scanned subject), and pre-positioning is not needed, thereby avoiding unnecessary radiation exposure to the patient, reducing the frequency of supplementary scanning, and improving the scanning efficiency.

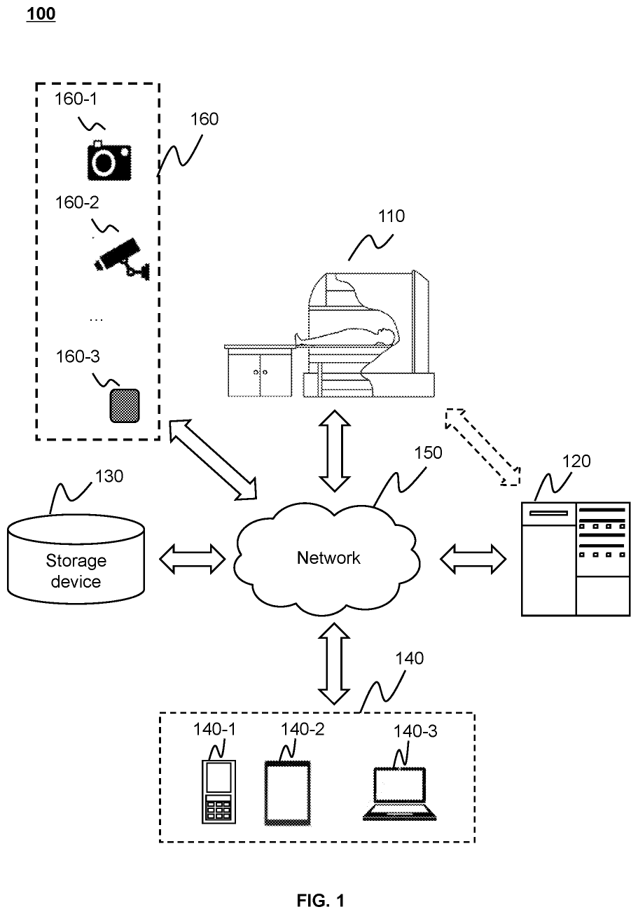

FIG. 1 is a schematic diagram illustrating an exemplary medical imaging system 100 according to some embodiments of the present disclosure. As shown in FIG. 1, in some embodiments, the medical imaging system 100 may include an imaging device 110, a processing device 120, a storage device 130, a terminal 140, a network 150, and a camera system 160.

The imaging device 110 may be configured to obtain image data of a subject. In some embodiments, the imaging device 110 may be a medical imaging device capable of imaging a specific portion (e.g., an ROI) of a patient. For example, the imaging device 110 may include a computed tomography (CT) scanner, a magnetic resonance imaging (MRI) scanner, a positron emission tomography (PET) scanner, or the like, or any combination thereof. The imaging device 110 provided above is for illustrative purposes only and is not limited in scope. In some embodiments, the imaging device 110 may include multiple different types of medical imaging devices for capturing multiple images of different modalities.

In some embodiments, the imaging device 110 may send the acquired image to the processing device 120. The imaging device 110 may receive instructions sent by doctors through the terminal 140, and perform related operations based on the instructions, such as scanning and imaging. In some embodiments, the imaging device 110 may exchange data and/or information with other components of the medical imaging system 100 (e.g., the processing device 120, the storage device 130, and the terminal 140) via the network 150. In some embodiments, the imaging device 110 may be directly connected to other components of the medical imaging system 100. In some embodiments, the imaging device 110 may include one or more components (e.g., the processing device 120 and the storage device 130) of the medical imaging system 100.

The processing device 120 may process information and/or data relating to the medical imaging system 100 to perform one or more functions described in the present disclosure. For example, the processing device 120 may classify multiple images into multiple groups. As another example, the processing device 120 may obtain a first target image of a target subject. The first target image may be taken by a camera system (e.g., the camera system 160). The processing device 120 may determine a marker in the first target image. The marker is configured to characterize a scanning position. The processing device 120 may determine a scanning range of a medical imaging device for scanning the target subject based on the marker.

The processing device 120 may be a single server or a server group. The server group may be centralized, or distributed (e.g., the processing device 120 may be a distributed system). In some embodiments, the processing device 120 may be local or remote. For example, the processing device 120 may access information and/or data stored in the imaging device 110 and/or the storage device 130 via the network 150. As another example, the processing device 120 may be directly connected to the imaging device 110 and/or the storage device 130 to access stored information and/or data. In some embodiments, the processing device 120 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the processing device 120 may include one or more sub-processing devices (e.g., a single-core processing device or multi-core processing device). Merely by way of example, the processing device 120 may include a central processing unit (CPU), a specialized integrated circuit (ASIC), a specialized instruction processor (ASIP), a graphics processor (GPU), a physical processor (PPU), a digital signal processor (DSP), a field programmable gate array (FPGA), a programmable logic circuit (PLD), a controller, a microcontroller unit, a reduced instruction set computer (RISC), a microprocessor, or any combination thereof.

The storage device 130 may store data or information generated by other devices. In some embodiments, the storage device 130 may store data and/or instructions that the processing device 120 may execute or use to perform exemplary methods described in the present disclosure. In some embodiments, the storage device 130 may include a mass storage device, a removable storage device, a volatile read-and-write memory, a read-only memory (ROM), or the like, or a combination thereof. In some embodiments, the storage device 130 may be implemented on a cloud platform. In some embodiments, the storage device 130 may be part of the imaging device 110, the processing device 120, and/or the terminal 140.

The terminal 140 may be configured to enable a user interaction between a user and the medical imaging system 100. In some embodiments, the terminal 140 may be connected to and/or communicate with the imaging device 110, the processing device 120, and/or the storage device 130. In some embodiments, the terminal 140 may be one or any combination of other devices with display screen, input and/or output functions, such as mobile devices 140-1, tablet computers 140-2, laptop computers 140-3, desktop computers, or the like. In some embodiments, the terminal(s) 140 may be part of the processing device 120 and/or the imaging device 110.

The network 150 may connect various components of the system and/or connect the system with external resource parts. The network 150 may enable communication between various components and with other parts outside the system, promoting the exchange of data and/or information. In some embodiments, one or more components of the medical imaging system 100 (e.g., the imaging device 110, the processing device 120, the storage device 130, and the terminal 140) may send data and/or information to other components via the network 150. In some embodiments, the network 150 may be any one or more of wired or wireless networks.

The camera system 160 may be configured to obtain one or more images (the term “image” herein refers to a single image or a frame of a video). In some embodiments, the camera system 160 may include a camera 160-1, a video recorder 160-2, an image sensor 160-3, etc. In some embodiments, the camera system 160 may include at least one camera device. For example, the camera system 160 may include one or more cameras. In some embodiments, the camera system 160 may capture images of the subject and transmit the captured images to one or more components (e.g., the processing device 120, the storage device 140) of the medical imaging system 100 via the network 150.

It should be noted that the above description is provided for illustrative purposes only and is not intended to limit the scope of the present disclosure. For ordinary technical personnel in this field, various changes and modifications can be made under the guidance of the content of the present disclosure. The features, structures, methods, and other features of the exemplary embodiments described in the present disclosure can be combined in various ways to obtain additional and/or alternative exemplary embodiments. In some embodiments, the medical imaging system 100 may include one or more additional components and/or one or more components described above may be omitted. Additionally or alternatively, two or more components of the medical imaging system 100 may be integrated into a single component. For example, the processing device 120 may be integrated into the imaging device 110. As another example, a component of the medical imaging system 100 may be replaced by another component that can implement the functions of the component. However, these changes and modifications will not deviate from the scope of the present disclosure.

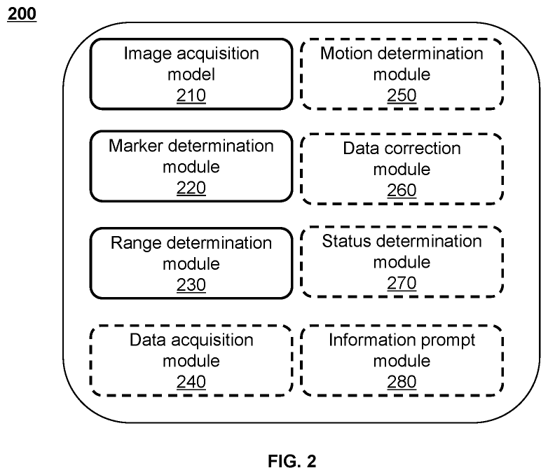

FIG. 2 is a schematic diagram illustrating an exemplary medical imaging system according to some embodiments of the present disclosure. As shown in FIG. 2, a medical imaging system 200 may include an image acquisition module 210, a marker determination module 220, and a range determination module 230. In some embodiments, each module of the medical imaging system 200 may be implemented by the processing device 120.

The image acquisition module 210 may be configured to obtain a first target image of a target subject. The first target image may be taken by a camera system (e.g., the camera system 160). More descriptions regarding the obtaining of the first target image may be found elsewhere in the present disclosure (e.g., operation S310 and the descriptions thereof).

The marker determination module 220 may be configured to determine a marker in the first target image. The marker may be configured to characterize a scanning position. More descriptions regarding the determination of the marker may be found elsewhere in the present disclosure (e.g., operation S320 and the descriptions thereof).

The range determination module 230 may be configured to determine a scanning range of a medical imaging device (e.g., the imaging device 110) for scanning the target subject based on the marker. More descriptions regarding the determination of the scanning range may be found elsewhere in the present disclosure (e.g., operation S330 and the descriptions thereof).

In some embodiments, the medical imaging system 200 may further include a data acquisition module 240, a motion determination module 250, and a data correction module 260.

The data acquisition module 240 may be configured to control a movement of a scanning bed, and in response to determining that the scanning range matches an imaging position, control the medical imaging device to scan the target subject. More descriptions regarding controlling the movement of the scanning bed may be found elsewhere in the present disclosure (e.g., operation S1110 and the descriptions thereof). More descriptions regarding controlling the medical imaging device to scan the target subject may be found elsewhere in the present disclosure (e.g., operation S1120 and the descriptions thereof).

The data acquisition module 240 may be further configured to acquire scanning data of the scanning range of the target subject when the medical imaging device scans the target subject. More descriptions regarding acquiring the scanning data may be found elsewhere in the present disclosure (e.g., operation S1130 and the descriptions thereof).

The image acquisition module 210 may be further configured to obtain at least two second images taken by the camera system during the process of scanning the target subject using the medical imaging device. More descriptions regarding obtaining at least two second images taken by the camera system may be found elsewhere in the present disclosure (e.g., operation S1140 and the descriptions thereof).

The motion determination module 250 may be configured to determine motion change information of the target subject based on the at least two second images. More descriptions regarding the determination of the motion change information may be found elsewhere in the present disclosure (e.g., operation S1150 and the descriptions thereof).

The data correction module 260 may be configured to perform artifact correction on the scanning data based on the motion change information to obtain a corrected scanning image. More descriptions regarding obtaining the corrected scanning image may be found elsewhere in the present disclosure (e.g., operation S1160 and the descriptions thereof).

In some embodiments, the medical imaging system 200 may further include a status determination module 270 and an information prompt module 270.

The image acquisition module 210 may be further configured to obtain a target depth image taken by the camera system. More descriptions regarding the obtaining a target depth image taken by the camera system may be found elsewhere in the present disclosure (e.g., operation S1310 and the descriptions thereof).

The status determination module 270 may be configured to determine an operation status of the camera system according to at least one of the target depth image and a target difference image. The target difference image is a difference between the target depth image and a historical depth image. The operation status may include at least one of a fault status, an abnormal tendency status, or a normal status. More descriptions regarding the determination of the operation status of the camera system may be found elsewhere in the present disclosure (e.g., operation S1320 and the descriptions thereof).

The information prompt module 270 may be configured to send prompt information to a user based on the operation status of the camera system. More descriptions regarding sending prompt information may be found elsewhere in the present disclosure (e.g., operation S1330 and the descriptions thereof).

It should be noted that the above descriptions of the medical imaging system 200 are provided for the purposes of illustration, and are not intended to limit the scope of the present disclosure. For persons having ordinary skills in the art, various modifications and changes in the forms and details of the application of the above system may occur without departing from the principles of the present disclosure. In some embodiments, the medical imaging system 200 may include one or more other modules and/or one or more modules described above may be omitted. Additionally or alternatively, two or more modules may be integrated into a single module and/or a module may be divided into two or more units. However, those variations and modifications also fall within the scope of the present disclosure.

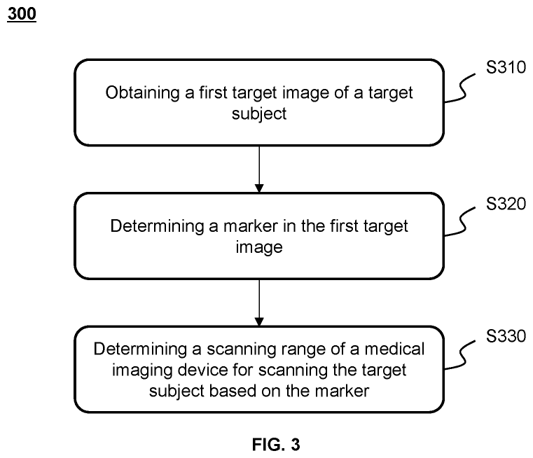

FIG. 3 is a flowchart illustrating an exemplary medical imaging method according to some embodiments of the present disclosure. In some embodiments, a process 300 may be executed by the medical imaging system 100 and/or the medical imaging system 200. For example, the process 300 may be implemented as a set of instructions (e.g., an application) stored in a storage device (e.g., the storage device 130 illustrated in FIG. 1). In some embodiments, the processing device 120 of the medical imaging system 100 and/or one or more modules of the medical imaging system 200 may execute the set of instructions and may accordingly be directed to perform the process 300. In the following description, the medical imaging system 100 is described as a CT system, where the imaging device 110 is a CT scanner.

In S310, a first target image of a target subject may be obtained. In some embodiments, the operation S310 may be performed by the image acquisition module 210 shown in FIG. 2.

Examination position of the target subject refers to a body posture of the target subject lying on the scanning bed during the scan. Exemplary examination positions include: HFP (Head First Prone, meaning the head is near the front of the medical imaging device and the face is down), HFS (Head First Supine, meaning the head is near the front of the medical equipment and the face is up), FFP (Feet First Prone, meaning the feet are near the front of the medical equipment and the face is down), FFS (Feet First Supine, meaning the feet are near the front of the medical equipment and the face is up), HFDR (Head First Decubitus Right, meaning the head is near the front of the medical equipment, lying on the side, with the right shoulder down), HFDL (Head First Decubitus Left, meaning the head is near the front of the medical equipment, lying on the side, with the left shoulder down), FFDR (Feet First Decubitus Right, meaning the feet are near the front of the medical equipment, lying on the side, with the right shoulder down), FFDL (Feet First Decubitus Left, meaning the feet are near the front of the medical equipment, lying on the side, with the left shoulder down). The above are just some typical examination positions, but actual applications are not limited to these listed positions. The present disclosure is illustrated by taking the examination position of the face being up or down (e.g., HFP, HFS, FFP, and FFS) as an example. It should be noted that the present disclosure is also applied in other examination positions.

The target subject is a subject that needs to be scanned. For example, the target subject may include a patient, a person to be examined, an animal, or the like, that needs to be scanned. The present disclosure is hereinafter described with the target subject being a patient. In some embodiments, the processing device 120 may take an image of the target subject through a camera system (e.g., the camera system 160) as the first target image. Taking the medical imaging system 100 as an example, the imaging device 110 may include a scanning gantry and a scanning bed, and the camera system 160 may be disposed inside of a scanning cavity of the scanning gantry, and/or outside of the scanning cavity such as on a ceiling or a wall of a scanning room, etc. The camera system 160 may receive an image acquisition instruction sent by the user terminal 140 to take the first target image including the target subject, and send the acquired first target image to the processing device 120. In some embodiments, the first target image may include a real-time image of the target subject. For example, the camera system may take a real-time video or a real-time image. Each video frame of the real-time video may be equivalent to a real-time image.

Merely by way of example, when the target subject is to be scanned, the target subject may be first moved onto a scanning bed, and then moved into the scanning cavity of the scanning gantry by moving the scanning bed. The scanning cavity may include at least one camera device of the camera system. The target subject may be scanned by the at least one camera device in the scanning cavity.

In some embodiments, the camera system may include one or more camera devices. For example, the camera system may include one, two, or three camera devices. Each of the one or more camera devices may be one of a monocular camera, a binocular camera, a structured light camera, or any combination thereof. In some embodiments, the camera system may include at least one of a first camera device and a second camera device mounted inside the scanning cavity. The first camera device may be disposed at a top of the scanning cavity of the medical imaging device, and the second camera device may be disposed on a side of the scanning cavity. In some embodiments, the camera system may include at least one camera device disposed at other positions in the scanning cavity. In some embodiments, the camera system may include at least one camera device disposed outside the scanning cavity. For example, the camera system may include a third camera device disposed on the ceiling of the scanning room above the scanning bed, and/or a fourth camera device disposed on the wall of the scanning room at the same level as the scanning bed. Through the one or more camera devices located at multiple different positions, images of the patient from multiple different viewing angles can be obtained, which facilitates the user to comprehensively and accurately observe the posture and position of the patient.

In some embodiments, the first target image may be captured by at least one camera device mounted inside the scanning cavity when at least a portion of the target subject is located in the scanning cavity. The first target image may include an image of at least one angle (i.e., viewing angle) of the target subject. For example, the first target image may include a first image of a top view of the target subject in the scanning cavity taken by the first camera device. As another example, the first target image may include a second image of a side view of the target subject in the scanning cavity taken by the second camera device. Based on the first image of the top view of the target subject and the second image of the side view of the target subject, the user can more clearly observe the posture and position of the target subject in the scanning cavity.

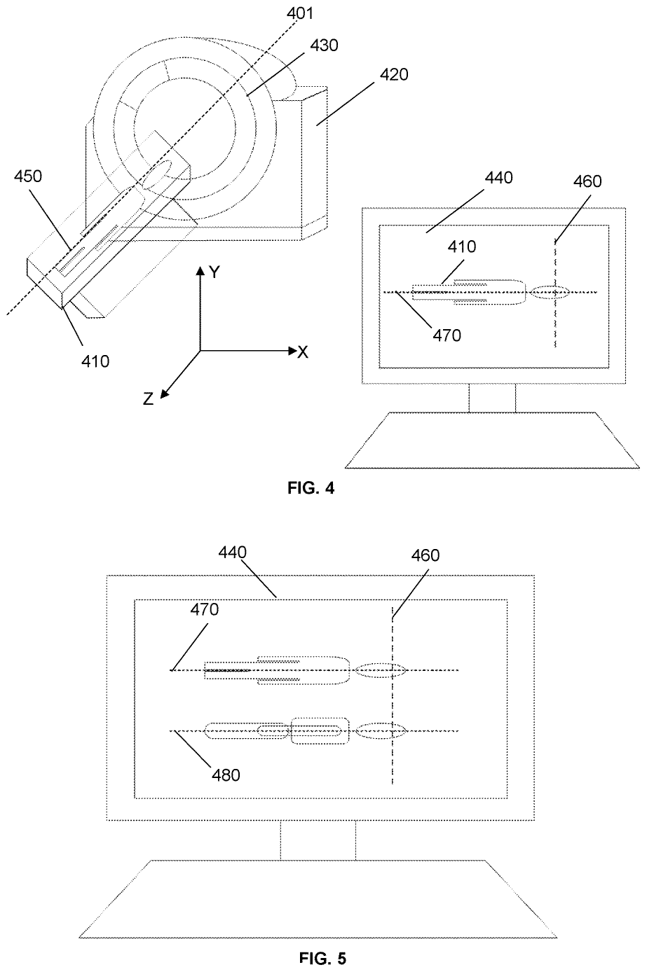

FIG. 4 is a schematic diagram illustrating an exemplary medical imaging system in operation according to some embodiments of the present disclosure. The medical imaging system shown in FIG. 4 may include a scanning bed 410, a scanning gantry 420, and a display 440. The scanning gantry 420 includes a scanning cavity 430. The medical imaging system shown in FIG. 4 may represent the medical imaging system 100, and the scanning bed 410 and the scanning gantry 420 may be a portion of the imaging device 110, and the display 440 may be a portion of the user terminal 140. As shown in FIG. 4, a target subject 450 may be located on the scanning bed 410. When the scanning bed 410 is moved, the target subject 450 may be moved into the scanning cavity 430. In this case, one or more camera devices of a camera system mounted within the scanning cavity 430 may take a first target image of the target subject 450, and the first target image may be displayed on a display interface of the display 440.

Merely by way of example, the camera system may include a first camera device mounted on the top of the scanning cavity 430 within the scanning cavity 430. As shown in FIG. 4, an image displayed on the display 440 is a first image of a top view of the target subject 450 taken by the first camera device.

As another example, the imaging system may include a first camera device and a second camera device. FIG. 5 is a schematic diagram illustrating a display interface of an exemplary medical imaging system according to some embodiments of the present disclosure. As shown in FIG. 5, an image displayed on an upper portion of the display 440 is a first image of a top view of the target subject 450 taken by the first camera device mounted on the top of the scanning cavity 430 within the scanning cavity 430, and an image displayed on a lower portion of the display 440 is a second image of a side view of the target subject 450 taken by the second camera device mounted on the side of the scanning cavity 430 within the scanning cavity 430.

In some embodiments, when the camera system includes at least one camera device mounted outside the scanning cavity and at least one camera device mounted inside the scanning cavity, the display may display images (also referred to as external image) captured by the at least one camera device mounted outside the scanning cavity and images (also referred to as internal image) captured by the at least one camera device mounted inside the scanning cavity. For example, the display interface of the display may include two portions. One portion is configured to display the internal image and the other portion is configured to display the external image. In this way, the user can observe the scanned subject more comprehensively and clearly.

In S320, a marker may be determined in the first target image. In some embodiments, the operation S320 may be performed by the marker determination module 220 shown in FIG. 2.

The marker is a marker of a feature in an image related to scanning. The marker may include a solid or dashed line segment, an ellipse, a circle, or a rectangle, etc. In some embodiments, the marker may be configured to characterize a scanning position. The marker may include at least one of an imaging position, a center line of the target subject, or a region of interest (ROI) of the target subject, etc. The imaging position may be configured to characterize a position on the target subject capable of being scanned by the medical imaging device (e.g., the imaging device 110). The center line may be configured to characterize a center line of a body of the target subject. The region of interest (ROI) of the target subject may include the head, chest, or abdomen, or the like, of the target subject.

Taking CT scanner as an example, in some embodiments, the imaging position may be a position on the target subject capable of being currently scanned by radiation of the medical imaging device. A radiation source may be provided on the scanning gantry. The radiation source may be disposed at a preset position to perform radiation scanning within a fixed range (e.g., the field of view (FOV) of the medical imaging device) corresponding to the preset position. When the target subject passes through the fixed range, a part of the target subject within the fixed range may be scanned by radiation. The imaging position may be equivalent to the part of the target subject within the fixed range.

In some embodiments, the processing device 120 may determine the imaging position according to a scanning protocol. The scanning protocol may be a set of scanning parameters used by the medical imaging device during scanning. For example, the scanning parameters may include at least one of a radiation exposure dose, scanning time, a count of uses, a scanning range, a scanning voltage, a scanning current, and a scanning mode (e.g., plain scanning, enhanced scanning, etc.) of radiation. Merely by way of example, after the first target image is obtained, the processing device 120 may determine the imaging position according to the scanning protocol and bed position information of the scanning bed to mark the imaging position in the first target image. The spatial position of the FOV of the medical imaging device may be determined based on the scanning protocol. It can be understood that during the movement of the scanning bed, since the spatial position of the FOV of the medical imaging device is fixed, and the spatial position of the moving scanning bed is changed in real time, the position on the target subject capable of being currently scanned by radiation of the medical imaging device changes in real time, and the imaging position also changes in real time, and the imaging position in the first target image also changes accordingly. By determining the imaging position based on the scanning protocol and the bed position information of the scanning bed, the position on the target subject capable of being currently scanned by radiation can be accurately determined, thereby ensuring the accuracy and reliability of the determined imaging position.

Merely by way of example, the processing device 120 may establish a global three-dimensional (3D) coordinate system (also referred to as spatial coordinate system) (e.g., the X-Y-Z coordinate system shown in FIG. 4) based on any point in a 3D space as an origin, and then determine a coordinate range of the scanning bed in the coordinate system, and a coordinate range corresponding to the fixed range in the coordinate system. The processing device 120 may determine a target coordinate range of the target subject on the scanning bed within the fixed range based on coordinates of each position on the scanning bed. The processing device 120 may map the target coordinate range to the first target image to mark the imaging position in the first target image.

As another example, the processing device 120 may determine a positional relationship between one or more camera devices and the scanning bed through shooting angles of the one or more camera devices of the camera system and an image of the scanning bed taken by the one or more camera devices. Since positions of the one or more camera devices and position of the fixed range are determined, the positions of the one or more camera devices and the position of the fixed range may be relatively fixed. The processing device 120 may map a positional relationship between the position of the scanning bed and the position of the fixed range based on a positional relationship between the positions of the one or more camera devices and the position of the fixed range, and then determine the position of the scanning bed capable of being currently scanned, i.e., the imaging position. More descriptions regarding specific embodiments of how to determine the imaging position may be found in FIG. 7.

In some embodiments, the imaging position in the first target image may be represented as a point, a line, or a region. For example, the position on the target subject capable of being currently covered by a center of the fixed range (e.g., the FOV of the medical imaging device) may be used as the imaging position and marked in the first target image. In this case, the imaging position in a first target image a top view of the target subject may be represented as a point. As another example, the position on the target subject capable of being currently covered by a center line of the fixed range may be used as the imaging position and marked in the first target image. The center line of the fixed range is a horizontal straight line, passes through the center of the fixed range, and is vertical to the axis direction of the scanning cavity. In this case, the imaging position in a first target image a top view of the target subject may be represented as a line (e.g., 460 shown in FIG. 5). As still another example, the position on the target subject capable of being currently covered by the fixed range may be used as the imaging position and marked in the first target image. In this case, the imaging position in a first target image a top view of the target subject may be represented as a region.

In some embodiments, the processing device 120 may identify the target subject in the first target image, and determine the center line of the target subject according to the identified target subject in the first target image. Specifically, the processing device 120 may identify the target subject in the first target image through an image recognition model or algorithm, extract local feature points representing body parts of the target subject through a feature extraction model or algorithm, and determine the center line of target subject according to the local feature points. Merely by way of example, the processing device 120 may train the image recognition model and the feature extraction model using a plurality of human body (e.g., a human body 3D model, and a real human body) images as training samples, and annotate the human body images with body parts, local feature points, and center lines as training labels through manual annotation or other modes. The processing device 120 may extract the local feature points of the target subject through the feature extraction model or algorithm, and determine the center line of the target subject according to the local feature points. The center line of the target subject extends along the height or length direction of the target subject. By determining the center line of the target subject according to the identified target subject in the first target image, the accuracy of the determined center line can be improved.

In some embodiments, during the movement of the scanning bed, the center line of the target subject may be aligned with an axis (e.g., 401 shown in FIG. 4) of the scanning gantry. The processing device 120 may identify the center line of the target subject in the first target image. The processing device 120 may display the first target image on a display device and display the center line of the target subject in the first target image. Merely by way of example, as shown in FIGS. 4-6, the processing device 120 may display a first image of a top view of the target subject in the scanning cavity on the display device, identify the center line of the target subject in the first image, and display the center line of the target subject in the first image (e.g., the center line 470 shown in FIGS. 4-6). When controlling the movement of the scanning bed in the scanning cavity, the user may use the displayed center line in the first image as a reference to align the center line of the target subject with the axis of the scanning cavity, so as to improve the positioning accuracy. For example, the center line displayed in the first image of a top view of the target subject may be used as a reference to align the center line of the target subject with the axis of the scanning cavity in the horizontal direction.

As another example, as shown in FIG. 5, the processing device 120 may display a second image of a side view of the target subject in the scanning cavity on the display device, identify the center line of the target subject in the second image, and display the center line of the target subject in the second image (e.g., the center line 480 shown in FIG. 5). When controlling the movement of the scanning bed in the scanning cavity, the user may use the displayed center line in the second image as a reference to align the center line of the target subject with the axis of the scanning cavity, so as to improve the positioning accuracy. For example, the center line displayed in the second image of a side view of the target subject may be used as a reference to align the center line of the target subject with the axis of the scanning cavity in the vertical direction.

In some embodiments, the processing device 120 may identify the axis of the scanning cavity in the first target image and display the axis of the scanning cavity in the first target image, so as to facilitate the aligning of the center line of the target subject with the axis of the scanning cavity. For example, the processing device 120 may obtain coordinates of the axis of the scanning cavity in the spatial coordinate system. The processing device 120 may obtain a transformation relationship between the spatial coordinate system and an image coordinate system. The transformation relationship may be determined by camera calibration of the camera system. The processing device 120 may determine coordinates of the axis of the scanning cavity in the image coordinate system based on the coordinates of the axis of the scanning cavity in the spatial coordinate system and the transformation relationship, thereby identifying the axis of the scanning cavity in the first target image.

In some embodiments, the processing device 120 may identify the target subject in the first target image. The processing device 120 may acquire scanning information. The scanning information indicates the ROI to be scanned of the target subject. For example, the scanning information may include text, image, audio and video information identifying the ROI of the target subject. The ROI of the target subject may include at least a portion of the target subject. For example, the ROI of the target subject may include heart, head, left chest, or right shoulder, etc. The processing device 120 may mark, based on the scanning information and the identified target subject in the first target image, a contour of the ROI in the first target image. For example, the first target image may include a full-body image of the target subject, and the processing device 120 may first identify a plurality of body parts (e.g., an external part such as head, hand, arm, foot, leg, neck, breast, abdomen, joint, etc., and/or an internal organ such as heart, lung, liver, stomach, kidney, etc.) in the full-body image, and then select a corresponding body part from the identified body parts according to the ROI indicated in the scanning information, and mark the contour of the ROI in the first target image. By marking the contour of the ROI in the first target image, the user can identify the ROI of the target subject, thereby more accurately determining the scanning range and better performing the scanning operation.

In some embodiments, the processing device 120 may mark the contour of the ROI in the first target image using a recognition model. The recognition model may be a machine learning model, such as a neural network model. An input of the recognition model may include the first target image and the scanning information, and an output of the recognition model may include the first target image with the marked contour of the ROI. The recognition model may be trained in the following manner. The processing device 120 may pre-collect a plurality of sample camera images presenting at least a portion of a human body (e.g., a 3D human body model, and an image presenting a real human body) as training samples. Each of the plurality of sample camera images may be marked by the user with a contour of a body part as a training label of the sample camera image. Information (e.g., name) of the marked body part may be used as sample scanning information corresponding to the sample camera image. Training may be performed based on the sample scanning information, and the plurality of sample camera images before and after the marking. The sample scanning information and the sample camera images before the marking (without the marking) may be input into a preliminary model. An output of the preliminary model and the sample camera images after the marking (with the marking) may be used to determine a loss function. By using the machine learning model to mark the contour of the ROI, the marking accuracy and the processing speed are improved.

The shape, size, and position of the internal organs of different people are different. For the recognition model that is trained only based on the sample camera images and the sample scanning information, the marked contour of an internal organ in the first target image output by the recognition model may have an offset from an actual contour of the internal organ of a specific patient. In some embodiments, in addition to sample camera images and sample scanning information of a sample subject, at least one of a medical history and a historical medical image of the ROI of the sample subject may be used as an input to train the preliminary model to obtain the recognition model. Accordingly, the input of the recognition model may further include at least one of a medical history of the target subject and a historical medical image of the ROI of the target subject. By including the medical history of the target subject and/or the historical medical image of the ROI of the target subject in the input of the machine learning model, the features of the target subject are considered, such that an output marking result is more targeted and accurate.

In S330, a scanning range of a medical imaging device for scanning the target subject is determined based on the marker. In some embodiments, the operation S330 may be performed by the range determination module 230 shown in FIG. 2.

In some embodiments, after the marker is determined, the processing device 120 may display the first target image including the marker. For example, the processing device 120 may display the first target image including the marker through a display interface of a display device (e.g., the display 440). As shown in FIGS. 4-6, the marker of the first target image displayed on the display interface of the display 440 may include a marked imaging position 460, a center line 470 and a center line 480 of the target subject 450, and an ROI 485 of the target subject 450. It should be noted that in addition to the target subject, the scanning bed and other background patterns are also present in the first target image acquired. In FIGS. 2-4, in order to facilitate the characterization of the positional relationship between the marker and the human body, only the target subject in the first target image is shown in the figures. It should be clear to those skilled in the art that the embodiment in which only the target subject is displayed in the visual interface and the embodiment in which the whole scene within the FOV of the camera system is directly displayed in the visual interface should be included in the protection scope of the present disclosure.

The scanning range of the target object is configured to make a reconstructed image include the whole ROI, and the position of the scanning range of the target object is the same or substantially the same as that of the ROI. The position of the scanning range of the target object being substantially the same as that of the ROI refers that a difference between the sizes along the movement direction (e.g., the Z direction shown in FIG. 4) of the scanning range and the ROI is less than a difference threshold (e.g., 1%, 2%, 5%, 10%, etc., of the size along the movement direction of the ROI) and the proportion of a portion of the ROI that is located outside the scanning range is less than a proportion threshold (e.g., 1%, 2%, 5%, 10%, etc.). For example, the scanning bed codes corresponding to the ROI and the scanning range are 1203-1303 mm. As another example, the scanning bed code corresponding to the ROI is 1203-1303 mm, and the scanning bed code corresponding to the scanning range is 1200-1306 mm. As still another example, the scanning bed code corresponding to the ROI is 1203-1303 mm, and the scanning bed code corresponding to the scanning range is 1206-1300 mm. In some embodiments, the processing device 120 may determine the scanning range of the target subject based on the displayed marker. For example, the processing device 120 may determine the scanning range of the target subject based on the displayed marker and user operation information. The user operation information may include bed operation information and/or confirmation operation information of the user. The bed operation information and the confirmation operation information may include an instruction issued by the user. The bed operation information may include a bed movement instruction, and the confirmation operation information may include a confirmation instruction for the user to confirm a start position and an end position. As another example, the processing device 120 may automatically determine the scanning range of the target subject based on the displayed marker. By determining the scanning range of the target subject based on the displayed marker, the accuracy of the determined scanning range can be improved, so as to be easier to operate, which simplifies the user operation and improves the scanning efficiency.

In some embodiments, the user may determine whether to move the bed according to the displayed marker. When the bed needs to be moved, the user may issue the bed movement instruction, the imaging device 110 may move the scanning bed according to the bed movement instruction, and the processing device 120 may determine the position information of the scanning bed after the movement according to the bed movement instruction.

For example, the center line of the target subject and the axis of the scanning cavity may be displayed in the first target image. The scanning bed may be controlled to move to align the center line of the target subject with the axis of the scanning cavity. As another example, the imaging position and the contour of the ROI of the target subject may be displayed in the first target image. The scanning bed may be controlled to move to align the center of the contour of the ROI of the target subject with the center of the imaging position. In some embodiments, the user may manually control, based on the displayed marker, the scanning bed to move. For example, the user can observe the real-time relative position between the center line of the target subject and the axis of the scanning cavity through the first target image. The user may manually control, through, e.g., a control rod or a button, the scanning bed to move while observing the real-time change of the relative position between the center line of the target subject and the axis of the scanning cavity through the first target image, until the center line of the target subject is aligned with the axis of the scanning cavity in the first target image. In some embodiments, the processing device 120 may automatically control, based on the displayed marker, the scanning bed to move. For example, the processing device 120 may determine a position difference between the center line of the target subject and the axis of the scanning cavity in the first target image. The processing device 120 may obtain a transformation relationship between the spatial coordinate system and an image coordinate system. The processing device 120 may determine a position difference between the center line of the target subject and the axis of the scanning cavity in the actual space by transforming the position difference between the center line of the target subject and the axis of the scanning cavity in the first target image using the transformation relationship. The processing device 120 may control the scanning bed to move based on the position difference in the actual space. In some embodiments, a real-time position difference between the center line of the target subject and the axis of the scanning cavity in the actual space may be marked and displayed in the first target image.

In the present disclosure, the X axis, the Y axis, and the Z axis shown in FIG. 4 may form an orthogonal coordinate system. The X axis and the Z axis shown in FIG. 4 may be horizontal, and the Y axis may be vertical. As illustrated, the positive X direction along the X axis may be from the left side to the right side of the medical imaging device seen from the direction facing the front of the medical imaging device; the positive Y direction along the Y axis shown in FIG. 4 may be from the lower part to the upper part of the medical imaging device; and the positive Z direction along the Z axis shown in FIG. 4 may refer to a direction in which the scanning bed is moved out of the scanning cavity of the medical imaging device. In some embodiments, the scanning bed may move along at least one of the X axis, the Y axis, and the Z axis shown in FIG. 4.

In some embodiments, the processing device 120 may determine the scanning range of the target subject according to real-time images of the target subject during a movement process of the scanning bed of the medical imaging device. More descriptions regarding the real-time image may be found in the related descriptions of the first target image in the operation S310. Specifically, during the movement process of the scanning bed, the processing device 120 may determine the scanning range of the target subject according to a real-time relative position relationship between the target subject and the imaging position.

The processing device 120 may determine the scanning range of the target subject according to a start position and an end position. The start position is a first target position related to the scanning bed, and the end position is a second target position related to the scanning bed. In some embodiments, the scanning range of the target subject may be a region between the start position and the end position. Therefore, determining the scanning range of the target subject may be equivalent to determining the start position and the end position. The start position and the end position may be displayed in the first target image.

In some embodiments, the start position and the end position may be characterized by a scanning bed code. For example, the scanning bed code refers to a scale marked along the length of the scanning bed, representing positions on the scanning bed along the length direction of the scanning bed. The scanning bed code may be marked in a way that increases incrementally from the head to the foot of the scanning bed, or vice versa, from the foot to the head of the scanning bed. For example, if the length of the scanning bed is 2 meters, the scanning bed code may be marked in millimeters as 0 millimeters to 2000 mm from the head to the foot of the scanning bed.

As another example, the scanning bed code of the start position may be set to zero, and the scanning bed code of the end position may be set relative to the start position. For example, the scanning bed code of the start position may be set to zero, and the scanning bed code of the end position that is 300 mm away from the start position may be set to 300 mm. Setting a zero scanning bed code for the starting position facilitates easy recording of the start position and the end position.

The complete ROI needs to be included between the start position and the end position. Meanwhile, in order to prevent the patient from receiving too much radiation, a range between the start position and the end position should not be too large. Therefore, it is very important to determine the start position and the end position.

In some embodiments, during the movement process of the scanning bed, the processing device 120 may receive a confirmation instruction. The confirmation instruction may include a confirmation instruction of a start position and a confirmation instruction of an end position. The confirmation instruction may be sent by the user or automatically sent by the processing device 120.

In some embodiments, the imaging position may be displayed in the first target image, and the contour or the position of the ROI of the target subject may not be determined and displayed in the first target image. A relationship between the imaging position and the target subject may be easily determined based on the first target image marked with the imaging position. In some embodiments, during the movement process of the scanning bed, the processing device 120 may mark the imaging position in the first target image in real time, such that the relationship between the imaging position and the target subject can be obtained in real time. An appropriate start position and an appropriate end position can be determined during the movement process of the scanning bed based on the relationship between the imaging position monitored in real time and the target subject, thereby improving the accuracy of the determined scanning range.

Merely by way of example, the movement process of the scanning bed may be controlled by the user. The user may clearly observe a current imaging position based on the displayed first target image marked with the imaging position. The user may move the scanning bed by sending the bed movement instruction to change the imaging position. During the movement process of the scanning bed, the processing device 120 may display the first target image marked with the imaging position in real time through a display device (e.g., the display 440), such that when the imaging position reaches an expected start position, the user may confirm that the current imaging position is the start position by sending the confirmation instruction of the start position; when the imaging position reaches an expected end position, the user may confirm that the current imaging position is the end position by sending the confirmation instruction of the end position. Specifically, a medical imaging system (e.g., the medical imaging system 100) may include a control box for the scanning bed. The user may send the bed movement instruction through the control box. After receiving the bed movement instruction, the processing device 120 may send an instruction to a control device of the scanning bed according to the bed movement instruction to instruct the control device of the scanning bed to move the scanning bed according to the bed movement instruction. During the movement process of the scanning bed, the one or more camera devices may take the first target image of the target subject in real time and mark a real-time imaging position in the first target image; and the user may send the confirmation instruction of the start position and the confirmation instruction of the end position through the control box according to the real-time imaging position to confirm the start position and the end position. The processing device 120 may read a scanning bed code corresponding to the start position and the end position based on the start position and the end position confirmed by the user, so as to determine the scanning bed code corresponding to the scanning range.

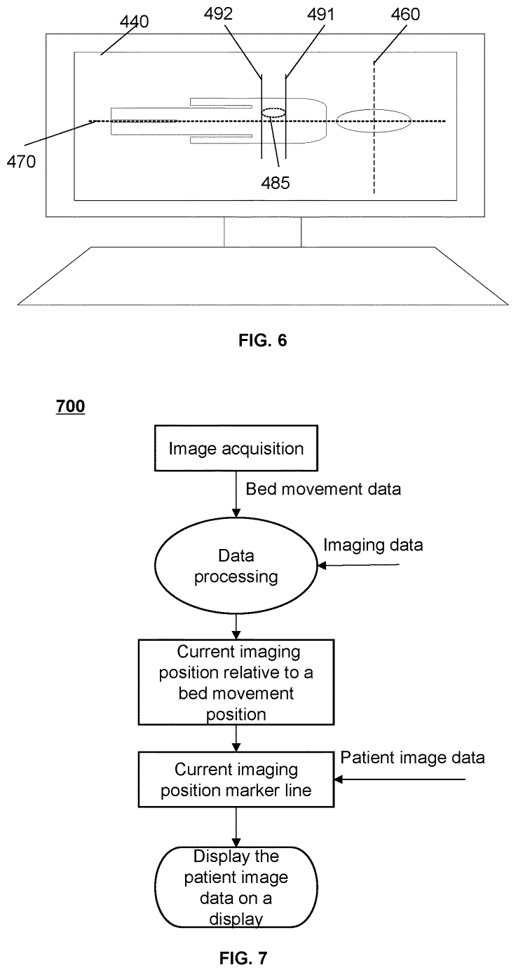

In some embodiments, the processing device 120 may determine and/or display the imaging position and the position of the ROI in the first target image, and determine the scanning range of the target subject based on the imaging position and the position of the ROI. The position of the ROI in the first target image may be automatically determined by the processing device 120 (e.g., as illustrated in operation S320), or the user may draw a contour of the ROI in the first target image to determine the position of the ROI in the first target image. Merely by way of example, during the movement process of the scanning bed, the user may determine the start position and the end position based on a boundary of the imaging position and the ROI displayed in the first target image. The user may issue the confirmation instruction of the start position when the current imaging position reaches a first target position related to the scanning bed (a first scanning bed code) that is deemed as a desired start position; and issue the confirmation instruction of the end position when the current imaging position reaches a second target position related to the scanning bed (a second scanning bed code) that is deemed as a desired end position. The processing device 120 may determine the first target position related to the scanning bed as the start position according to the confirmation instruction of the start position, and determine the second target position related to the scanning bed as the end position according to the confirmation instruction of the end position, so as to determine the scanning range of the target subject according to the start position and the end position. FIG. 6 is a schematic diagram illustrating another display interface of an exemplary medical imaging system according to some embodiments of the present disclosure. In FIGS. 6, 460 denotes a current imaging position, 470 denotes a center line of a target subject, 491 denotes a start position, 492 denotes an end position, and 485 denotes an ROI. As shown in FIG. 6, the current imaging position 460 does not reach a region corresponding to the ROI 485. In this case, the scanning bed may be controlled to continue moving such that the imaging position 460 continues moving. When the imaging position 460 moves to the position of the start position 491, the user may send a confirmation instruction of a start position to determine the position corresponding to 491 as the start position; when the imaging position 460 moves to the position of the end position 492, the user may send a confirmation instruction of an end position to determine the position corresponding to 492 as the end position.

As another example, during the movement process of the scanning bed, the processing device 120 may automatically determine the start position and the end position based on a boundary of the imaging position and the ROI determined in the first target image. In response to determining that the current imaging position reaches a first target position related to the scanning bed (a first scanning bed code) that is deemed as a desired start position, the processing device 120 may automatically determine the first target position related to the scanning bed as the start position. In response to determining that the current imaging position reaches a second target position related to the scanning bed (a second scanning bed code) that is deemed as a desired end position, the processing device 120 may automatically determine the second target position related to the scanning bed as the end position. The processing device 120 may determine the scanning range of the target subject according to the start position and the end position.

In some embodiments, the processing device 120 may determine at least one image position in the first target image by receiving a user instruction performed in the displayed first target image, and determine the scanning range of the target subject based on the at least one image position. The user instruction may include a marking instruction and a confirmation instruction. The marking instruction may be configured to mark the at least one image position in the first target image. The at least one image position may include a target imaging position desired by the user, i.e., the start position and the end position. By manually determining the scanning range of the target subject in the first target image by the user, the needs of the user can be better met, and the user operation can be further simplified.

Merely by way of example, the user may directly mark the start position and the end position in the displayed first target image through the marking instruction. For example, the user may mark certain positions in the first target image as the start position and the end position through a touch operation of a touch screen, an input operation of a mouse and a keyboard, a voice input, etc.

In some embodiments, the processing device 120 may automatically determine at least one image position in the first target image based on the position of the ROI of the target subject determined in the first target image, and determine the scanning range of the target subject based on the at least one image position. For example, the processing device 120 may determine, in the first target image, two outermost pixel points respectively at two ends of the contour of the ROI along a movement direction (e.g., the Z direction in FIG. 4) of the scanning bed. The processing device 120 may determine, in the first target image, two target pixels points that are respectively at a preset distance (e.g., 0 mm, 1 mm, 2 mm, etc.) from the two outermost pixel points and located outside the ROI. The processing device 120 may determine the scanning bed codes corresponding to the two target pixel points as the start position and the end position. Automatically determining the scanning range of the target subject on the first target image can further simplify user operation, greatly reduce the burden for the user, and thus improve the scanning efficiency.

Merely by way of example, the processing device 120 may determine the start position and the end position according to contour information of the ROI of the target subject in the first target image. Taking the display interface shown in FIG. 6 as an example, the start point 491 and the end point 492 are at two contour boundaries of the ROI 485 along the movement direction of the scanning bed. The processing device 120 may determine the two boundaries as the start position and the end position, respectively, so as to determine the scanning range of the target subject. It can be seen that the scanning range determined in this way includes the ROI 485.

Determining the start position and the end position in the first target image (e.g., by the user or the processing device 120) can be performed when the scanning bed keeps still in the scanning cavity, in which the process for controlling the scanning bed to move for determining the scanning range is omitted, thereby simplifying the process for determining the scanning range.

After the scanning range is determined, the scanning bed may be controlled to move to make the imaging position align with the start position. The processing device 120 may control the medical imaging device to scan the target subject in the scanning range from the start position. For example, the scanning bend may be controlled to move in the scanning range during the scan of the target subject. The processing device 120 may obtain imaging data during the scan of the target subject, and generate at least one medical image of the target subject by performing image reconstruction on the imaging data.

In the embodiments of the present application, the camera system takes a first target image, identifies and marks the features of the first target image such as the imaging position, center line, and the ROI of the target subject, and displays the marked first target image to the user, such that the user can easily observe the real-time imaging position and other conditions, and the user can determine the scanning range of the target subject before the actual scanning, thereby improving the accuracy of the determined scanning range and the scanning quality. By using image processing technology for determining the scanning range, pre-scanning for generating a scout image is not needed, which prevents the target subject from receiving unnecessary radiation, while reducing the frequency of re-scanning, and improving the scanning efficiency.

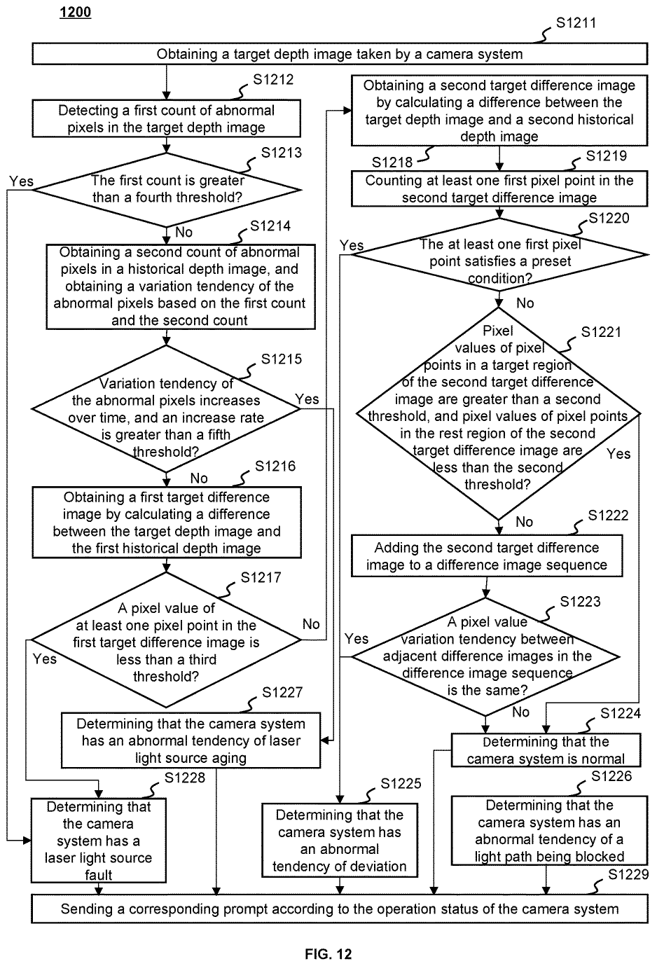

In some embodiments, before the camera system captures the first target image, the processing device 120 may obtain a target depth image taken by the camera system. The processing device 120 may determine an operation status of the camera system according to at least one of the target depth image and a target difference image. The processing device 120 may further send prompt information to the user based on the operation status of the camera system. In response to determining that the camera system operates normally, the processing device 120 may perform the process 300 to determine the marker and determine the scanning range. In response to determining that the camera system is in a fault status, the processing device 120 may send a prompt information of the fault status to the user. A camera device with a normal status may replace the camera device in the fault status. The processing device 120 may perform the process 300 with the new camera device. In response to determining that the camera system is in an abnormal tendency status, the processing device 120 may send a prompt information of the abnormal tendency status to the user, and perform the process 300. If the camera system has an abnormal tendency of deviation, the processing device 120 may determine an offset of the camera system using a second fault detection model. The processing device 120 may consider the offset of the camera system when transforming a position in the first target image into a position in space. More descriptions regarding determining an operation status of the camera system and sending prompt information based on the operation status may be found in FIG. 11 and FIG. 12.

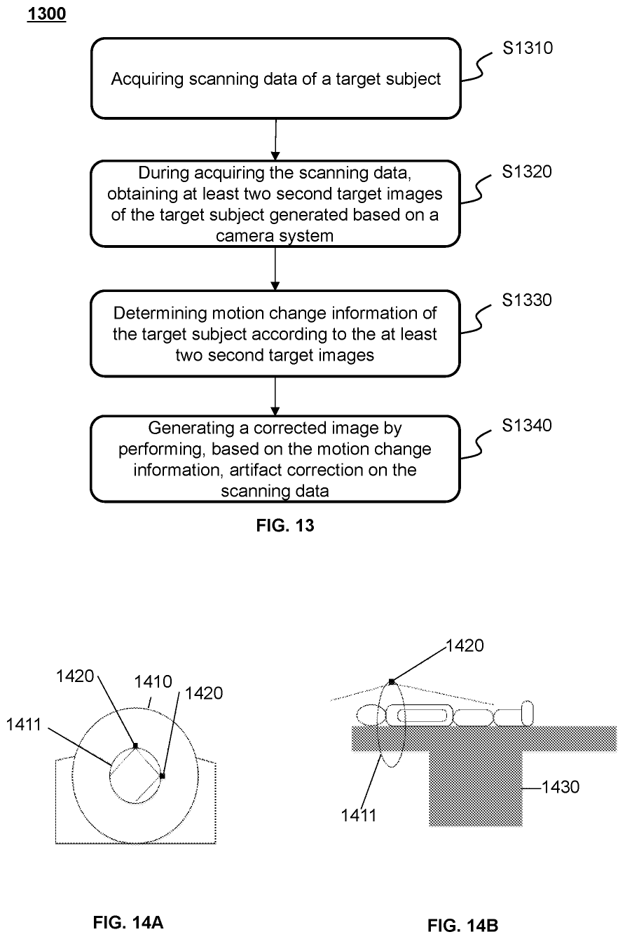

In some embodiments, During acquiring the imaging data (also referred to as scanning data), the processing device 120 may acquire least two second target images taken by the camera system at different time points. The processing device 120 may determine motion change information of the target subject according to the at least two second target images. The processing device 120 may further generate a corrected image by performing, based on the motion change information, artifact correction on the imaging data. More descriptions regarding generating the corrected image may be found in FIG. 13, FIG. 14A, and FIG. 14B.

FIG. 7 is a schematic diagram illustrating an exemplary medical imaging method according to some embodiments of the present disclosure. In some embodiments, at least part of a process 700 may be performed to achieve at least part of the process 300 as described in connection with FIG. 3. For example, the processing device 120 may determine a marker in a first target image (e.g., an image of a patient) by performing at least part of the process 700.

As shown in FIG. 7, the processing device 120 may obtain a first target image through a camera system (e.g., the camera system 160) to obtain bed movement data, imaging device data, and patient image data. The bed movement data may include a real-time position of a scanning bed. The imaging device data may include a real-time imaging position. The patient image data may include patient body image information. The processing device 120 may process the bed movement data and the imaging device data, determine a current imaging position according to a current gantry status, and determine a current imaging position relative to a bed movement position based on the obtained bed movement data. The processing device 120 may generate a patient body image according to the patient image data, generate a current imaging position marker line (the marker line is a line configured to mark the current imaging position, e.g., the marked imaging position 460) on the patient body image, and display the patient image data on a display (e.g., the display 440) of a user terminal (e.g., the user terminal 140), such that a doctor can easily obtain a relative position relationship between the current imaging position and the patient through the displayed image.

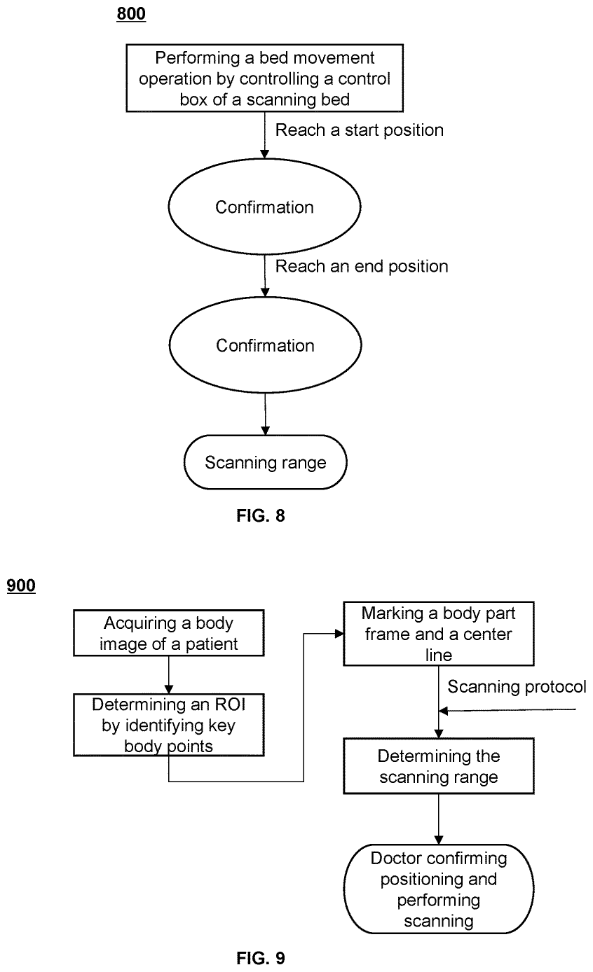

FIG. 8 is a schematic diagram illustrating an exemplary medical imaging method according to some embodiments of the present disclosure. In some embodiments, at least part of a process 800 may be performed to achieve at least part of the process 300 as described in connection with FIG. 3. For example, the processing device 120 may determine a scanning range of a medical imaging device for scanning a target subject based on a marker by performing at least part of the process 800. In some embodiments, the method shown in the process 800 may be performed after the process 700.

As shown in FIG. 8, the doctor may perform a bed movement operation by controlling a control box of a scanning bed in the medical imaging system (e.g., the medical imaging system 100). During the movement process of the scanning bed, a display (e.g., the display 440) of a user terminal (e.g., the user terminal 140) may always display a current imaging position, and the doctor may clearly observe a relationship between the current imaging position and a human body. When the doctor observes that the displayed imaging position reaches a start position, the doctor may perform confirmation (e.g., by sending a confirmation instruction of a start position) to mark the start position. When the doctor observes that the displayed imaging position reaches an end position, the doctor may perform confirmation (e.g., by sending a confirmation instruction of an end position) to mark the end position. The processing device 120 may determine the scanning range based on the start position and the end position confirmed by the user.

FIG. 9 is a schematic diagram illustrating an exemplary medical imaging method according to some embodiments of the present disclosure. In some embodiments, at least part of a process 900 may be performed to achieve at least part of the process 300 as described in connection with FIG. 3. For example, the processing device 120 may determine a scanning range based on a first target image (i.e., a patient body image) and perform scanning by performing at least part of the process 900.

As shown in FIG. 9, after a doctor confirms an ROI of a patient, the patient may be moved into a scanning cavity through a scanning bed, and the processing device 120 may acquire a body image of the patient through a camera system (e.g., the camera system 160) disposed on a scanning gantry, and acquire a real-time patient body image in the scanning cavity as a first target image. The processing device 120 may identify key body points based on the acquired patient body image, perform iterative estimation of body parameters to determine a current ROI in the scanning cavity, and mark a body part frame and a center line on the patient body image. The camera system may include a first camera device disposed at a top of the scanning cavity in the scanning cavity and a second camera device disposed on a side of the scanning cavity in the scanning cavity. The first camera device may be configured to take a first target image of a top view of the patient, and a center line of the first target image of the top view marks a sagittal plane and a cross section of the ROI. The second camera device may be configured to take a second image of a side view of the patient, and a center line of the second image of the side view marks a coronal plane of the ROI. The processing device 120 may determine the scanning range based on the marked body part frame and the center line, and information of the ROI in a scanning protocol. The scanning protocol may include the information of the ROI. The processing device 120 may display a real-time patient body image in the scanning cavity and the marked body part frame and the center line to the doctor on the display (e.g., display 440) of the user terminal (e.g., the user terminal 140), such that the doctor can directly observe the body part of the patient and the position thereof in the scanning cavity. The doctor may adjust the position of the patient and the scanning range to align a center of the body with a center of the scanning cavity. After the doctor confirms the positioning, the scanning may be performed.

In the embodiments of the present disclosure, the body part image of the patient is taken by the camera system of the medical imaging device, and the frame and the center line of the ROI are automatically detected and marked, assisting the doctor in positioning the patient and determining the scanning range, thereby simplifying and speeding up the patient positioning process, improving the accuracy of the scanning range, and enhancing the scanning positioning accuracy and the scanning quality.

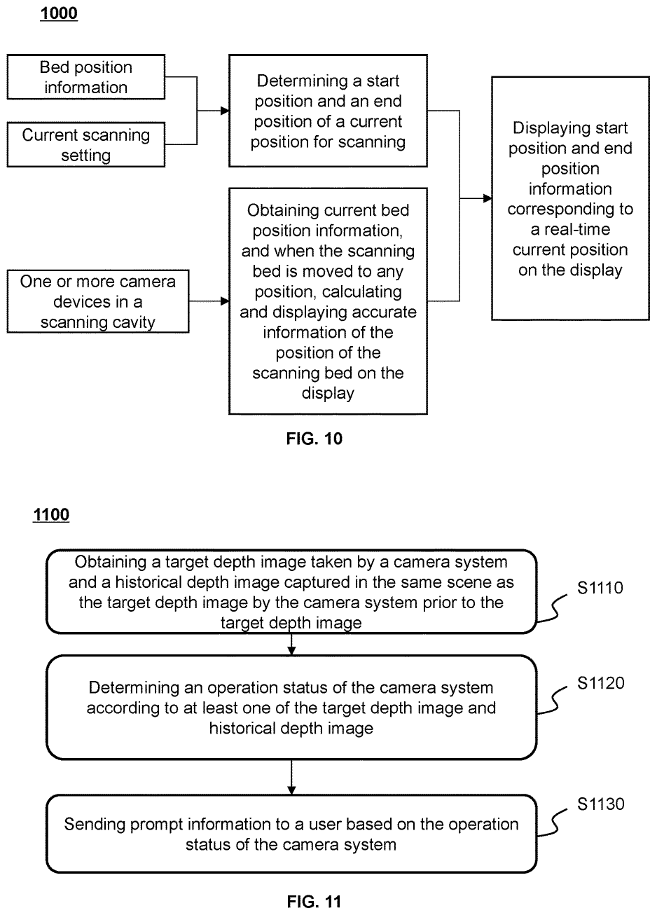

FIG. 10 is a schematic diagram illustrating an exemplary medical imaging method according to some embodiments of the present disclosure. In some embodiments, at least part of a process 1000 may be performed to achieve at least part of the process 300 as described in connection with FIG. 3. For example, the processing device 120 may determine a scanning range of a target subject based on a displayed marker by performing at least part of the process 1000.

As shown in FIG. 10, in order to determine the scanning range, the processing device 120 may determine a start position and an end position corresponding to the current imaging position according to bed position information and a current scanning setting. A region between the start position and the end position may be a current scanning range. The scanning setting may include a scanning type, etc. The scanning type may include Helical, Axial, etc. The processing device 120 may acquire an image through one or more camera devices (e.g., a first camera device) in the scanning cavity to obtain current bed position information. When the scanning bed is moved to any position, the processing device 120 may calculate and display accurate information of the position of the scanning bed on the display. The processing device 120 may display start position and end position corresponding to a real-time current imaging position or bed position on the display for the doctor to view. During the movement process of the scanning bed, the processing device 120 may determine a new imaging position in the next image of a certain image according to the imaging position and the bed position information determined in the certain image. Determining a new imaging position is equivalent to performing a coordinate transformation based on the previous image, which makes it possible to simply and quickly calculate the position of the marker during the movement process of the scanning bed, so as to display the marker in time.

During use of the medical imaging system, the camera device is usually used to complete functions such as auxiliary positioning, collision avoidance, calibration, and real-time monitoring of a patient status. At present, hardware faults of the camera device is usually determined manually. For example, a customer service engineer views, using third-party software, an image captured by the camera device through a user interface prompt to determine a specific damaged part of the camera device caused by the hardware fault, which requires a long time between the occurrence of an abnormality in the hardware of the camera device and the confirmation of the abnormality of the camera device. In addition, during this period, erroneous images provided by the camera device affect operation of other devices in the medical imaging system, resulting in the inability to use the medical imaging system normally.

The embodiments of the present disclosure provide a medical imaging method and system. The medical imaging method may comprise obtaining a target depth image taken by a camera system. The camera system may be mounted in a medical device operation room. The medical imaging method may comprise determining an operation status of the camera system according to at least one of the target depth image and a target difference image. The target difference image may be a difference between the target depth image and a historical depth image. The operation status may include a fault status, an abnormal tendency status, or a normal status. The medical imaging method may further comprise sending prompt information to a user based on the operation status of the camera system.

According to the embodiments of the present disclosure, by determining the determining the operation status of the camera system according to at least one of the target depth image and a target difference image, and sending the prompt information to the user based on the operation status of the camera system, the detection of abnormalities of the camera system is no longer dependent on manual work, which shortens the time required between the occurrence of an abnormality in the camera system and the confirmation of the abnormality, and can detect an abnormal tendency, thereby facilitating the maintenance of the camera system and ensuring the normal operation of the medical imaging system.

FIG. 11 is a flowchart illustrating an exemplary medical camera detection according to some embodiments of the present disclosure. In some embodiments, a process 1100 may be executed by the medical imaging system 100 and/or the medical imaging system 200. For example, the process 1100 may be implemented as a set of instructions (e.g., an application) stored in a storage device (e.g., the storage device 130 illustrated in FIG. 1). In some embodiments, the processing device 120 of the medical imaging system 100 and/or one or more modules of the medical imaging system 200 may execute the set of instructions and may accordingly be directed to perform the process 1100. In some embodiments, the processing device 120 may determine an operation status of a camera system by executing the method shown in process 1100, and send prompt information based on the operation status.

In the present disclosure, determining an operation status of one depth camera is taken as an example. When a camera system includes two or more depth camera, the processing device 120 may determine an operation status of each depth camera according to the camera detection method of the present disclosure (e.g., the process 1100 and/or the process 1200).

In S1110, a target depth image taken by a camera system and a historical depth image captured in the same scene as the target depth image by the camera system prior to the target depth image may be obtained. In some embodiments, the operation S1110 may be performed by the image acquisition module 210 shown in FIG. 2.

A depth image is an image used to represent depth information. The target depth image refers to a depth image currently being processed, and is also referred to as a current depth image. A pixel value of each pixel in the depth image is used to represent the depth information. The depth information is a distance between an object corresponding to the pixel in a 3D environment and a camera device that takes the image. The depth image may be taken using a camera device that is a depth camera in the camera system.

Depth cameras can be divided into a depth camera based on binocular stereo vision, a depth camera based on structured light, and a depth camera based on time of flight (ToF) according to the imaging principle. The following describes the operation principles of each type of depth camera.

The principle of the depth camera based on binocular stereo vision is similar to that of human eyes. The images seen by the left eye and the right eye are different. The position of the same object seen by the left eye and the right eye is different. This difference is called parallax, which is inversely proportional to the distance of the object from the human eyes. According to this principle, the depth camera based on binocular stereo vision includes two cameras with a known relative position relationship. After matching the corresponding points of the images taken by the two cameras together, a parallax image between the two images can be obtained. Then the depth image can be obtained by combining the internal parameters of the camera and the relative position relationship. Under an ideal condition, the distance from the object to the camera corresponding to a certain pixel in the depth image to the camera can be expressed as z=b*f/x, where b denotes the distance between the two cameras, f denotes the focal length of the camera, and x denotes the calculated parallax. The depth camera based on binocular stereo vision does not need to use a laser light source.

The depth camera based on structured light requires a laser light source. The light emitted by the light source is transformed into structured light with uneven light and dark distribution by a projector and then projected out. The camera, as a receiving end, collects the structured light reflected by objects in the environment. Affected by objects in the environment, the light and dark distribution in the structured light is distorted, and this distortion reflects the distance between the object and the depth camera. The depth image can be calculated by combining this distortion with the distance between the light source and the camera.

The above two types of depth cameras directly output the depth image. The depth image is a two-dimensional (2D) image, in which a pixel value of each pixel represents the depth information of the pixel, i.e., distance information from the object corresponding to the pixel to the depth camera.

The depth camera based on ToF also requires a laser light source. The basic principle is that the laser light source emits a laser pulse, and a sensor acts as a receiving end to detect the laser reflected back by an object. A difference between emission time and reception time of the laser is the ToF, and a product of the ToF and the speed of light is the distance of the object. Direct use of this basic principle requires high accuracy in time measurement. In practical applications, the laser light source is usually modulated with continuous waves, and the distance of the object is calculated based on a phase difference between the laser emitted by the light source and the received laser. This type of depth camera directly outputs point cloud data. The point cloud data includes 3D coordinates of multiple points each of which represents a point on the surface of the object in an actual environment. The point cloud data can be converted into a depth image for subsequent processing.

In practical applications, the depth camera can be implemented based on various principles. For example, the structured light can be combined with the binocular vision, i.e., two cameras with fixed relative positions are used to collect the reflected structured light.

In some embodiments, a camera system (e.g., the camera system 160) may include a depth camera. The process 1100 is described by taking one or more camera devices of the camera system being depth cameras as an example. One or more depth cameras may be mounted in a medical device (e.g., the imaging device 110) operation room. Specific mounting position of the one or more depth cameras may include a ceiling and a wall of the medical device operation room, a position inside a scanning gantry, etc., which are not limited in the present disclosure. Merely by way of example, the one or more depth camera in the camera system may be based on the principle of structured light and/or ToF, i.e., the one or more depth cameras need to use a laser light source.

In some embodiments, the processing device 120 may take the target depth image through the one or more depth cameras in the camera system. The one or more depth camera may take a depth image of a moving object as the target depth image. For example, the processing device 120 may take a depth image of a target subject through the one or more depth cameras as the target depth image. As another example, the processing device 120 may take a depth image of a scanning bed through the one or more depth camera as the target depth image.

In S1120: an operation status of the camera system may be determined according to at least one of the target depth image and the historical image. In some embodiments, the operation S1120 may be performed by the status determination module 270 shown in FIG. 2.

The processing device 120 may generate a target difference image by determining a difference between the target depth image and the historical depth image, and determine the operation status of the camera system based on the target difference image.

The depth cameras based on principle of structured light and ToF both need to use the laser light source, and obtain the depth information of the object by collecting laser light reflected back from the object. The farther the object is from the depth camera, the weaker the reflected laser light. When the reflected laser light is too weak to be received by the receiving end, the depth camera cannot detect the object. Therefore, the depth cameras based on principle of structured light and ToF are limited in measurement range in terms of principle. It is understood that the greater the power of the laser light source, the larger the measurement range. In addition, the laser light reflected back from the object mainly comes from diffuse reflection. If the object has a certain degree of transparency, or is smooth enough to be close to a mirror, the object also has an adverse effect on the reflection of the laser, making it difficult for the depth camera to detect the object.

Abnormal pixels refer to pixels with abnormal pixel values in the depth image. For example, the distance of a portion where the receiving end of the depth camera cannot collect the reflected laser light cannot be calculated. A pixel value of a pixel corresponding to this situation in the depth image can be set to a specific value to indicate no depth, and this pixel is an abnormal pixel. In addition to no depth, abnormal pixels can also include pixel points (“pixel” and “pixel point” can be used interchangeably in the present disclosure) whose pixel values indicate a depth of 0. In some embodiments, the pixel value representing a depth of 0 and the pixel value representing no depth may be the same, e.g., the pixel value of 0. In some embodiments, the processing device 120 may determine the abnormal pixels based on the pixel values of pixels in the depth image.

The target difference image is a difference between the target depth image and a historical depth image. A pixel value of each pixel in the target difference image is a pixel value of the pixel in the target depth image minus a pixel value of the same pixel in the historical depth image. The historical depth image is a depth image collected in the same scene as the target depth image by the camera system prior to the target depth image. For example, the historical depth image may be collected when the depth camera is just mounted. The operation status of the camera system is a description of a current operation status of the depth camera, which is used to reflect whether the depth camera operate normally. The operation status may include at least one of a fault status, an abnormal tendency status, or a normal status. The fault status may include a light source fault, a motion mechanism fault, etc. In some embodiments, the light source fault may include a laser light source fault, etc. The fault status means that the depth camera can no longer operate properly and the collected depth image can no longer be used. The abnormal tendency status may include a deviation of the depth camera, blocking of a light path of the depth camera, aging of the laser light source of the depth camera, etc. In the case of the abnormal tendency status, the collected depth image can still be used temporarily, but there may be certain errors. Accordingly, the errors may be further expanded and need to be processed.

In some embodiments, the processing device 120 may add the target difference image to a difference image sequence including at least one historical difference image. The at least one historical difference image may be obtained using the historical depth image as a subtrahend image. The at least one historical difference image in the difference image sequence may be arranged in an order in which the at least one historical difference image is added to the difference image sequence. In response to determining that a pixel value variation tendency between adjacent difference images in the difference image sequence is the same, the processing device 120 may determine that the camera system has an abnormal tendency of deviation.

Merely by way of example, since an image can be represented in a matrix form, the process of calculating the target difference image can be implemented by matrix subtraction. The process may be represented by the following formula:

D=C−H (1),

where D denotes the target difference image, C denotes the target depth image, and H denotes the historical depth image. C is a minuend matrix in matrix subtraction, and is also referred to as a minuend mage. H is a subtrahend matrix in matrix subtraction, and is also referred to as a subtrahend image.

In some embodiments, the at least one historical difference image in the difference image sequence may be obtained using the same historical depth image as the subtrahend image, and may be arranged in an order in which the at least one historical difference image is added to the difference image sequence. The order in which the at least one historical difference image is added to the difference image sequence may be an order in which the corresponding target depth image as the minuend image is acquired or processed.

In some embodiments, the target depth image and the historical depth image include the same target subject, and the target subject may be a movable object (e.g., a scanning bed) or a fixed object. A position and/or a posture of the target subject in the historical depth image and a position and/or a posture of the target subject in the target depth image may be the same. Merely by way of example, in order to obtain the target difference image, when the target depth image is taken, the processing device 120 may make the position and/or the posture of the target subject as consistent with the position and/or the posture of the target subject in the historical depth image as possible. Since the position and/or the posture of the target subject remains consistent in the target depth image and the historical depth image, theoretically the pixel values of difference images are also consistent. In fact, due to the influence of factors such as noise, there is a certain measurement error, so pixel values of adjacent difference images may vary. Due to the randomness of noise, the variation is random. If the same variation tendency appears (e.g., continuous increase or continuous decrease), it means that the depth camera may have an abnormal tendency of deviation.

Taking difference images D1-D3 arranged in time order as an example, for a pixel location, the pixel value of a pixel P1 at the pixel location in D1 is V1, the pixel value of a pixel P2 at the pixel location in D2 is V2, and the pixel value of a pixel P3 at the pixel location in D3 is V3. The processing device 120 may determine whether V1, V2, and V3 are increased or decreased in sequence. In response to determining that for each of more than a certain proportion (50%, 60%, 80%, 100%, etc.) of the pixel positions, the pixels values corresponding to the pixel position in D1-D3 are increased in sequence, or for each of more than a certain proportion (50%, 60%, 80%, 100%, etc.) of the pixel positions, the pixels values corresponding to the pixel position in D1-D3 are decreased in sequence, the processing device 120 determines that a pixel value variation tendency in the difference image sequence is the same.

In some embodiments, in response to determining that at least one first pixel point in the target difference image satisfies a preset condition, the processing device 120 may determine that the camera system has an abnormal tendency of deviation. The position and/or the posture of the target subject in the historical depth image and the position and/or the posture of the target subject in the target depth image may be the same. The processing device 120 may determine whether the at least one first pixel point in the target difference image satisfies the preset condition based on a single target difference image. The at least one first pixel point may be at least one pixel of which a pixel value is greater than a first threshold (e.g., 5, 7, 8, etc.). The preset condition may include that a count of the at least one first pixel point is greater than a preset value (e.g., 500, 1000, etc.). Whether the camera system has the abnormal tendency of deviation is determined based on whether the count of the at least one pixel point with the pixel value greater than the first threshold is greater than the preset value, such that the influence of measurement errors can be eliminated, thereby improving the determination accuracy of the operation status of the camera system.

In an ideal condition, when the historical depth image and the target depth image are taken, the position and/or the posture of the target subject is exactly the same, and the historical depth image and the target depth image should be exactly the same, i.e., the pixel values of all pixel points in the target difference image are 0. In fact, due to the influence of factors such as noise, there is a certain measurement error, and the pixel value of the at least one pixel point in the target difference image may not be 0, but a smaller value close to 0. The pixel value of the at least one first pixel point in the target difference image being greater than the first threshold means that the at least one pixel point in the historical depth image and the at least one pixel point located at a corresponding position in the target depth image have a depth variation that exceeds a measurement error range. The first threshold is a threshold related to the pixel values of the pixel points in the difference image. In some embodiments, the first threshold may be determined based on the measurement error range and a depth image pixel value bit width. For example, the first threshold may be greater than an upper limit of the measurement error range. Assuming that the upper limit of the measurement error range is ±5, the first threshold may be set to 8, 10, etc.

In some embodiments, the preset condition may further include other conditions. For example, the preset condition may further include that a gradient exist in the target difference image. Whether the gradient exists may be determined based on the pixel values of the pixel points in the target difference image. If the pixel values show the same variation tendency (e.g., increase or decrease) along a specific direction, it is determined that the gradient exists.

For example, pixels in an image are arranged in rows and columns. Taking a row of pixels as an example, in response to determining that the pixels values of the row of pixels are increased in sequence, the processing device 120 may determine that the row of pixels is in an increasing tendency. In response to determining that the pixels values of the row of pixels are decreased in sequence, the processing device 120 may determine that the row of pixels is in a decreasing tendency. In response to determining that more than a certain proportion (50%, 60%, 80%, 100%, etc.) of rows of pixels are all in the increasing tendency, or all in the decreasing tendency, the processing device 120 may determine that the same variation tendency exists along the row direction, indicating that the gradient exists in the row direction. Determining that the gradient exists in the column direction is similar to determining that the gradient exists in the column direction.

In some embodiments, in response to determining that pixel values of pixel points in a target region of the target difference image are greater than a second threshold, and pixel values of pixel points in the rest region of the target difference image are less than the second threshold, the processing device 120 may determine that the camera system has an abnormal tendency of a light path being blocked. The light path refers to a path that is within the depth camera and between the lens and the sensor of the depth camera. The light path being blocked may include that there is a foreign object in the light path. The position and/or the posture of the target subject in the historical depth image and the position and/or the posture of the target subject the target depth image may be the same. Based on a single target difference image, the processing device 120 may determine whether the pixel values of the pixel points in the target region of the target difference image is greater than the second threshold, and whether the pixel values of the pixel points in the rest region is less than the second threshold. The second threshold is also a threshold related to the pixel values of the pixel points in the target difference image. The second threshold may be set in a similar manner to the first threshold. In some embodiments, the values of the second threshold and the first threshold may be close or even the same.

It is understood that in a region where the light path is blocked, the pixel values of the pixel points vary significantly, while in a region where the light path is not blocked, the pixel values of the pixel points do not vary significantly. The pixel values of the pixel points in the target difference image may reflect a variation in the pixel values from the historical depth image to the target depth image. By determining whether the camera system has an abnormal tendency of being blocked according to the pixel values of the pixel points in different regions of the target difference image, whether the light path is blocked can be accurately determined according to the variation in the pixel values of different regions in the target difference image, thereby improving the determination accuracy of the operation status of the camera system.

In some embodiments, the position and/or the posture of the target subject in the historical depth image and the position and/or the posture of the target subject in the target depth image may be different, and the processing device 120 may determine a posture variation and/or a position variation of the target subject according to movement control information of the target subject (e.g., a scanning bed). The processing device 120 may adjust at least one of the historical depth image and the target depth image based on the posture variation and/or the position variation of the target subject to reduce the influence caused by the movement of the target subject. For example, the processing device 120 may determine an affected region of at least one of the historical depth image and the target depth image according to the posture variation and/or the position variation of the target subject, set pixel values of pixel points in an affected region of the target difference image to 0, or modify the pixel values of the pixel points in the affected region of at least one of the historical depth image and the target depth image according to the posture variation and/or the position variation of the target subject. The affected region is a region of the image that is affected by the posture variation and/or the position variation of the target subject.

In some embodiments, the posture and/or the position of the target subject in the historical depth image and the posture and/or the position of the target subject in the target depth image may be different. In this case, in response to determining that pixel values of pixel points in the target difference image are less than or equal to a third threshold, the processing device 120 may determine that the camera system has a light source fault. For example, the processing device 120 may determine that the camera system has a laser light source fault. The third threshold is also a threshold related to the pixel values of the pixel points in the target difference image. In some embodiments, the third threshold may not be greater than the upper limit of the measurement error range. For example, the third threshold may be less than the first threshold and the second threshold.

Under normal circumstances, from the historical depth image to the target depth image, a depth of a region corresponding to the moving target subject varies significantly. The variation reflected on the target difference image means that there are pixels with relatively large pixel values in the target difference image. Merely by way of example, assuming that the third threshold is equal to the upper limit of the measurement error range, if the pixel values of the pixel points in the target difference image are greater than the third threshold, it means that the variation of the pixel values exceed the measurement error range, which can be considered to be caused by the movement of the target subject; if the pixel values of the pixel points in the target difference image are less than or equal to the third threshold, it means that the variation of the pixel values is within the measurement error range, and it can be considered that the pixel values do not vary actually, which is obviously different from the normal circumstances and indicates the light source fault. As set forth above, the pixel values of the pixel points in the target difference image can reflect whether the moving target subject exists. By determining whether the pixel values of the pixel points in the target difference image are less than the third threshold, whether the camera system has the light source fault is determined. The light source fault can be determined based on the moving target subject, which improves the determination accuracy of the operation status of the camera system.

In some embodiments, in response to determining that pixel values of all pixel points in the target difference image are less than or equal to the third threshold, the processing device 120 may determine that the camera system has a light source fault. In some embodiments, in response to determining that a proportion of pixels of which pixel values are less than or equal to the third threshold in the target difference image is greater than or equal to a count threshold (e.g., 50%, 60%, 70%, 80%, 90%, 100%, etc.), the processing device 120 may determine that the camera system has a light source fault. In some embodiments, the processing device 120 may identify an affected region in the target difference image. The affected region is a combination of a region of the target subject in the historical depth image and a region of the target subject in the target depth image. In response to determining that pixel values of all pixel points in the affected region in the target difference image are less than or equal to the third threshold, the processing device 120 may determine that the camera system has a light source fault.

In some embodiments, in response to determining that a count of abnormal pixels in the target depth image is greater than a fourth threshold, the processing device 120 may determine that the camera system has a light source fault. Details regarding the abnormal pixels can be found above. The fourth threshold may be a ratio threshold, a quantity threshold, etc. The ratio threshold refers to a proportion of abnormal pixels in the target depth image. The quantity threshold may be a product of the ratio threshold and a total count of pixels in the depth image. In some embodiments, the processing device 120 may set the fourth threshold according to an environment in which the depth camera is mounted and the objects therein. For example, when an indoor range is small and there are no or few objects (e.g., reflective objects, transparent objects, etc.) that affect laser reception in the mounting environment, the fourth threshold may be set to a positive number closer to 0 (e.g., the ratio threshold may be 0.03, 0.05, etc.); as the indoor range increases and the count of objects that affect laser reception in the mounting environment increases, the fourth threshold may increase accordingly.

It is understood that the abnormal pixels in the depth image cannot be used for the subsequent calculation of the target difference image. When a large number of abnormal pixels appear in the target depth image, it means that the target depth image as a whole cannot be used, which is often caused by the light source fault of the depth camera. For example, when the laser light source is not working, it may appear that all pixels in the target depth image are abnormal pixels. By determining whether the camera system has the light source fault based on whether the count of abnormal pixels in the target depth image is greater than the fourth threshold, the operation status of the laser light source can be accurately determined, thereby improving the determination accuracy of the operation status of the camera system.

In some embodiments, the processing device 120 may determine a variation tendency (e.g., increase, decrease, etc.) of the abnormal pixels based on the count of abnormal pixels in the target depth image and a count of abnormal pixels in the historical depth image. In response to determining that the variation tendency is increasing over time (e.g., the count of abnormal pixels in the target depth image is larger than the count of abnormal pixels in the historical depth image) and an increase rate is greater than a fifth threshold, the processing device 120 may determine that the camera system has an abnormal tendency of light source aging (e.g., laser light source aging). The increase rate is a ratio of a count difference to an interval between the target depth image and the historical depth image, and the count difference is a difference between the count of abnormal pixels in the target depth image and the count of abnormal pixels in the historical depth image. The fifth threshold is a threshold of variation rate (e.g., the increase rate). The fifth threshold may be determined based on a design life of the laser light source. For example, the processing device 120 may estimate the natural aging rate of the laser light source based on the design life, and then set the fifth threshold to a value greater than or equal to the natural aging rate to detect abnormal aging of the laser light source.

In practical applications, the laser light source is usually the part with the greatest loss in the one or more depth cameras. As the operation time increases, the laser light source gradually ages. One of the manifestations of aging is power reduction. As the power of the laser light source decreases, the measurement range also decreases, causing some objects that are originally measurable to become unmeasurable, which is manifested as the count of abnormal pixels increasing over time in the depth image. The fifth threshold is set to reduce false detection caused by measurement errors. By determining whether the camera system has the abnormal tendency of laser light source aging based on the variation tendency of the abnormal pixels, whether the probability of the laser light source is reduced can be accurately reflected, thereby determining the abnormal tendency of laser light source aging, and improving the determination accuracy of the operation status of the camera system.

In some embodiments, the processing device 120 may obtain an existence probability of each fault type (e.g., a light source fault, a motion mechanism fault, a deviation of the depth camera, blocking of a light path of the depth camera, aging of the laser light source of the depth camera, etc.) of the operation status by inputting the target depth image into a first fault detection model. The first fault detection model may be a machine learning model. For example, the first fault detection model may be a multi-classification model such as decision trees, naive Bayes, random forest, neural networks, etc. An input of the first fault detection model may include the target depth image, or a set of the target depth image and the historical depth image. An output of the first fault detection model may include the existence probability value of each fault type of the operation status. Training sample data of the first fault detection model may include a sample target depth image taken by one or more depth cameras in different fault status or a normal status, or a set of the sample target depth image and a sample historical depth image. Labels of the first fault detection model may include statuses corresponding to the one or more depth cameras that take the sample depth image, such as whether the one or more depth cameras have a fault, an abnormal tendency or is normal, and a type of the fault or the abnormal tendency. By determining the fault type through the machine learning model, the determination speed and accuracy of the fault can be improved, and a plurality of fault types can be determined at the same time, thereby greatly improving the determination efficiency and accuracy of the operation status of the camera system.

In some embodiments, for a fault type, in response to determining the existence probability of the fault type is greater than or equal to a second probability threshold (e.g., 80%, 90%, 95%, etc.), the processing device 120 may determine that a fault of the fault type exists in the camera system. In response to determining the existence probability of the fault type is less than or equal to a first probability threshold (e.g., 40%, 30%, 20%, 10%, 5%, etc.), the processing device 120 may determine that the camera system does not have a fault of the fault type. In some embodiments, for a fault type of which the existence probability is greater than the first probability threshold and less than the second probability threshold, the processing device 120 may determine a result indicating whether the fault type exists in the camera system by inputting the target depth image into a second fault detection model corresponding to the fault type for re-detection of the fault type. The second fault detection model may be a machine learning model. For example, the second fault detection model may be a binary classification model such as the decision trees or a support vector machine. An input of the second fault detection model may include the target depth image, or a set of the target depth image and the historical depth image. An output of the second fault detection model may include an existence probability of a specific fault type. Training sample data of the second fault detection model may include a sample target depth image taken by one or more depth cameras in a specific fault status or a non-specific fault status (i.e., a status in which the specific fault does not exist), or a set of the sample target depth image and a sample historical depth image. Labels of the second fault detection model may include statuses as to whether the specific fault exists in the one or more depth cameras that take the sample depth image. Re-detecting the specific fault type through the machine learning model can improve the detection speed and accuracy of the specific fault type, thereby greatly improving the determination efficiency and accuracy of the operation status of the camera system, while reducing the probability of fault detection errors, and improving the reliability of the detection result.

In some embodiments, the processing device 120 may detect a fault type of the operation status using at least two detection manners; and in response to determining that detection results obtained by the at least two detection manners are different, determine a result indicating whether the fault type exists in the camera system by inputting the target depth image into the second fault detection model corresponding to the fault type for re-detection of the fault type. For example, for an abnormal tendency of deviation, the processing device 120 may determine whether there is an abnormal tendency of deviation using the at least two detection manners, as described above. For example, the processing device 120 may determine whether there is an abnormal tendency of deviation based on a pixel value variation tendency between adjacent difference images in the difference image sequence; and the processing device 120 may determine whether there is an abnormal tendency of deviation based on whether the at least one first pixel point in the target difference image satisfies the preset condition. If the results of the two detection manners are contradictory, the processing device 120 may further determine whether there is an abnormal tendency of deviation using the second fault detection model corresponding to the abnormal tendency of deviation. By re-detecting the fault type with contradictory results using the machine learning model, the accuracy of fault detection is further improved.

In some embodiments, for the second fault detection for light source aging (e.g., the laser light source aging), the processing device 120 may determine an aging degree of a light source in the camera system using the second fault detection model. That is, the output of the second fault detection for light source aging may further include an aging degree of the light source in the camera system. Accordingly, during the training process of the second fault detection for light source aging, the labels may further include an aging degree of the light source used to take the sample depth image. By detecting the aging degree of the laser light source using the machine learning model, the detection speed and accuracy of the aging degree of the laser light source are improved.

In some embodiments, for the second fault detection model for abnormal tendency of deviation, the processing device 120 may determine an offset of the camera system using the second fault detection model. That is, the output of the second fault detection model for abnormal tendency of deviation may further include the offset of the camera system. Accordingly, during the training process of the second fault detection for abnormal tendency of deviation, the labels may further include an offset of the depth camera used to take the sample depth image.

In some embodiments, the camera system may be configured to determine a position of an object (e.g., a position of the scanning bed or the target subject) in the medical device operation room. The processing device 120 may obtain a positional relationship between the scanning bed and the camera system, and correct the positional relationship based on the offset of the camera system obtained using the second fault detection model. By correcting the positional relationship between the scanning bed and the camera system based on the offset of the camera system obtained based on the machine learning model, the correction effect is improved.

In some embodiments, if the processing device 120 determines that all faults and abnormalities do not exist in the camera system, it can be determined that the camera system operates normally.

In S1130, prompt information may be sent to a user based on the operation status of the camera system. In some embodiments, the operation S1130 may be performed by the information prompt module 280 shown in FIG. 2.

In some embodiments, the processing device 120 may send the detected operation status of the camera system to the user through prompt information. The prompt information may be implemented in various forms. For example, the prompt information may include one of a text, a sound, an image, an animation, a video, or the like, or any combination thereof, which is not limited in the present disclosure.

In the embodiments of the present disclosure, the operation status of the camera system is determined based on at least one of the target depth image and the target difference image, and the prompt information is sent to the user according to the operation status, such that the detection of abnormalities in the camera system is no longer dependent on manual work, which shortens the time required between the occurrence of an abnormality in the camera system and the confirmation of the abnormality, and can detect the abnormal tendency, facilitate the maintenance of the camera system, and ensure the normal operation of the medical imaging system. Meanwhile, by detecting the operation status of the camera system using the machine learning model, the detection speed and accuracy are improved, and the detection efficiency is improved.

FIG. 12 is a flowchart illustrating an exemplary camera detection method according to some embodiments of the present disclosure. In some embodiments, a process 1200 may be executed by the medical imaging system 100 and/or the medical imaging system 200. For example, the process 1200 may be implemented as a set of instructions (e.g., an application) stored in a storage device (e.g., the storage device 130 illustrated in FIG. 1). I n some embodiments, the processing device 120 of the medical imaging system 100 and/or one or more modules of the medical imaging system 200 may execute the set of instructions and may accordingly be directed to perform the process 1200. In some embodiments, the processing device 120 may determine an operation status of a camera system by performing the method shown in the process 1200, and send prompt information based on the operation status.

In S1211, a target depth image taken by a camera system may be obtained. The operation S1211 may may be found in the related descriptions of the operation S1110.

In S1212, a count of abnormal pixels in the target depth image may be detected.

In S1213, whether the count of the abnormal pixels is greater than a fourth threshold may be determined. In response to determining that the count of the abnormal pixels is greater than the fourth threshold, the processing device 120 may perform operation S1228; in response to determining that the count of the abnormal pixels is not greater than the fourth threshold, the processing device 120 may perform operation S1214.

In S1214, a count of abnormal pixels in a historical depth image may be obtained, and a variation tendency of the abnormal pixels may be obtained based on the count of the abnormal pixels in the target depth image and the count of the abnormal pixels in the historical depth image.

In S1215, whether the variation tendency of the abnormal pixels increases over time, and an increase rate is greater than a fifth threshold may be determined. In response to determining that the variation tendency of the abnormal pixels increases over time, and the increase rate is greater than the fifth threshold, the processing device 120 may perform operation S1226; in response to determining that the variation tendency of the abnormal pixels does not increase over time, or the increase rate is not greater than the fifth threshold, the processing device 120 may perform operation S1216.

In S1216, a first target difference image may be obtained by calculating a difference between the target depth image and a first historical depth image. The first historical depth image may be at least a portion of the historical depth image, and a posture and/or a position of the target subject in the first historical depth image may be different from the posture and/or the position of the target subject in the target depth image.

In S1217, whether pixel values of pixel points in the first target difference image are less than a third threshold may be determined. In response to determining that the pixel values of the pixel points in the first target difference image are less than the third threshold, the processing device 120 may perform operation S1228; in response to determining that the pixel values of the pixel points in the first target difference image are not less than the third threshold, the processing device 120 may perform operation S1218.

In S1218, a second target difference image may be obtained by calculating a difference between the target depth image and a second historical depth image. The second historical depth image may be at least a portion of the historical depth image, and the posture and/or the position of the target subject in the second historical depth image may be the same as the posture and/or the position of the target subject in the target depth image.

In S1219, at least one first pixel point in the second target difference image may be counted. The processing device 120 may determine, in the second target difference image, a count of the at least one first pixel point of which a pixel value is greater than a first threshold.

In S1220, whether the at least one first pixel point in the second target difference image satisfies a preset condition may be determined. The processing device 120 may determine whether the count of the at least one first pixel point in the second target difference image satisfies the preset condition. In response to determining that the at least one first pixel point in the second target difference image satisfies the preset condition, the processing device 120 may perform operation S1225; in response to determining that the at least one first pixel point in the second target difference image does not satisfy the preset condition, the processing device 120 may perform operation S1221. The preset condition may include that the count of the at least one first pixel point is greater than a preset value.

In S1221, whether pixel values of pixel points in a target region of the second target difference image are greater than a second threshold, and pixel values of pixel points in the rest region of the second target difference image are less than the second threshold is determined. The processing device 120 may determine whether the pixel values of the pixel points in the target region of the second target difference image are greater than the second threshold, and the pixel values of the pixel points in the rest region of the second target difference image are less than the second threshold. In response to determining that the pixel values of the pixel points in the target region of the second target difference image are greater than the second threshold, and the pixel values of the pixel points in the rest region of the second target difference image are less than the second threshold, the processing device 120 may perform operation S1226; in response to determining that the pixel values of the pixel points in the target region of the second target difference image are not greater than the second threshold, or the pixel values of the pixel points in the rest region of the second target difference image are not less than the second threshold, the processing device 120 may perform operation S1222.

In S1222, the second target difference image may be added to a difference image sequence.

In S1223, whether a pixel value variation tendency between adjacent difference images in the difference image sequence is the same may be determined. In response to determining that the pixel value variation tendency between the adjacent difference images in the difference image sequence is the same, the processing device 120 may perform operation S1225; in response to determining that the pixel value variation tendency between the adjacent difference images in the difference image sequence is not the same, the processing device 120 may perform operation S1224.

In S1224, it is determined that the camera system is normal. After the operation S1224, the processing device 120 may perform the operation S1229.

In S1225, it is determined that the camera system has an abnormal tendency of deviation. After the operation S1225, the processing device 120 may perform the operation S1229.

In S1226, it is determined that the camera system has an abnormal tendency of a light path being blocked. After the operation S1226, the processing device 120 may perform the operation S1229.

In S1227, it is determined that the camera system has an abnormal tendency of laser light source aging. After the operation S1227, the processing device 120 may perform the operation S1229.

In S1228, it is determined that the camera system has a laser light source fault. After the operation S1228, the processing device 120 may perform the operation S1229.

The operations S1212-S1228 may be found in the related descriptions of the operation S1120.

In S1229, a corresponding prompt may be sent according to the operation status of the camera system. The operation S1229 may be found in the related descriptions of the operation S1130.

The process 1200 illustrates an exemplary process for determining an operation status of the camera system using a combination of the detection manners described above. It should be noted that only one detection manner may be performed to determine an operation status of the camera system. Or at least two detection manners may be performed to determine an operation status of the camera system, and the order for performing the at least two detection manners is not limited. For example, the at least two detection manners may be performed in sequence or simultaneously.

In medical imaging, it is difficult for the ROI of the patient to remain completely still due to factors such as breathing and pathological convulsions. As a result, a scanned and reconstructed medical image may have motion artifacts, i.e., artifacts in the form of “blurring” and “ghosting” appear in the scanning image. The image motion artifacts reduce the image quality and the accuracy of the doctor's reading of the medical image, which in turn have a negative impact on the doctor diagnosis of the patient condition.

The embodiments of the present disclosure provide a medical imaging method and system. The medical imaging method may comprise acquiring scanning data of a target subject. The medical imaging method may comprise during acquiring the scanning data, obtaining at least two second target images taken by the camera system. The medical imaging method may comprise determining motion change information of the target subject according to the at least two second target images. The medical imaging method may further comprise generating a corrected image (also referred to as a corrected scanning image) by performing, based on the motion change information, artifact correction on the scanning data.

According to the embodiments of the present disclosure, the motion change information of the target subject is determined according to the at least two second target images taken by the camera system, and image artifact correction is performed on the scanning data of the medical imaging device based on the motion change information, thereby reducing the motion artifacts in the image and improving the quality and accuracy of medical imaging.

FIG. 13 is a flowchart illustrating an exemplary medical imaging method according to some embodiments of the present disclosure. In some embodiments, a process 1300 may be executed by the medical imaging system 100 and/or the medical imaging system 200. For example, the process 1300 may be implemented as a set of instructions (e.g., an application) stored in a storage device (e.g., the storage device 130 illustrated in FIG. 1). In some embodiments, the processing device 120 of the medical imaging system 100 and/or one or more modules of the medical imaging system 200 may execute the set of instructions and may accordingly be directed to perform the process 1300. In some embodiments, after the operation S330, the processing device 120 may control a movement process of the scanning bed, in response to determining that a scanning range matches an imaging position, control the medical imaging device to scan the target subject, and correct scanning data by performing the method shown in the process 1300.

In S1310, scanning data of a target subject acquired by scanning the target subject by a medical imaging device may be acquired. In some embodiments, the operation S1310 may be performed by the data acquisition module 240 shown in FIG. 2.

In some embodiments, the processing device 120 may acquire the scanning data of the target subject by scanning the target subject through the medical imaging device. More descriptions regarding the target subject may be found in FIG. 3. The scanning data refers to raw data that is acquired by the medical imaging device and configured to reconstruct a medical image. Taking CT device as an example, the scanning data refers to projection data detected by a detector of the CT device. Taking MR device as an example, the scanning data refers to K-space data. Taking PET device as an example, the scanning data refers to list-mode data or sinogram data.

In S3120, during acquiring the scanning data, at least two second target images generated based on a camera system may be obtained. The at least two second target images correspond to different time points. In some embodiments, the operation S1320 may be performed by the image acquisition module 210 shown in FIG. 2.

In some embodiments, during acquiring the scanning data, the processing device 120 may obtain the at least two second target images taken by the camera system (e.g., the camera system 160). In some embodiments, the processing device 120 may take images of the target subject at different viewing angles through one or more camera devices (e.g., a first camera device disposed at a top of a scanning cavity of the medical imaging device within the scanning cavity, and a second camera device disposed on a side of the scanning cavity within the scanning cavity) disposed in a scanning cavity of a gantry of the medical imaging device. One or more camera devices may be provided. For example, when one camera device is provided, the processing device 120 may change the view angle of the camera device for taking the image of the target subject by rotating the gantry relative to the scanning bed. As another example, when more camera devices are provided, the camera devices may be arranged at different angles around the scanning cavity. FIG. 14A and FIG. 14B are schematic diagrams illustrating a mounting position of a camera system of an exemplary medical imaging system according to some embodiments of the present disclosure. As shown in FIG. 14A and FIGS. 14B, 1430 denotes a scanning bed, 1410 denotes a front housing of a scanning gantry, and 1411 denotes a scanning cavity in the scanning gantry. Two camera devices 1420 may be disposed in the scanning cavity 1411. The two camera devices 1420 may be respectively located in a 0° direction and a 90°direction of a circumferential direction of the scanning cavity 1211. Any one of the two camera devices 1420 may be a 2D or 3D camera device. As shown in FIG. 14B, when a patient on the scanning bed 1230 is scanned using the medical imaging device, an image taken by the camera device 1420 located in the 0° direction is a coronal plane of the patient, and an image taken by the camera device 1420 located in the 90° direction is a sagittal plane of the patient.

In S1330, motion change information of the target subject may be determined according to the at least two second target images. In some embodiments, the operation S1330 may be performed by the motion determination module 250 shown in FIG. 2.

The motion change information refers to position change information of the target subject due to a movement of the target subject. The target subject may include a patient, a small animal, etc. In some embodiments, the motion change information may be configured to indicate motion information of an ROI of the target subject due to the movement. The motion change information may include motion status information and/or motion time information. The motion status information may include at least one of position change information of ROI of the target subject and/or orientation change information of the ROI. For example, the position change information may include position information (e.g., a position) of the ROI, a movement distance X, etc. As another example, the orientation change information may include orientation parameters (e.g., a pitch, a yaw, and a roll), a rotation angle θ, or the like, of the ROI. The motion time information may be configured to indicate a motion start time and/or a motion end time at which the ROI generates the motion change information, i.e., a start time (e.g., the motion start time) and an end time (e.g., the motion end time) at which the ROI generates the motion change information. With the motion status information and the motion time information, the motion change of the target subject can be determined from a spatial dimension and a time dimension at the same time, so as to more comprehensively and accurately reflect the motion change of the target subject.

In some embodiments, the at least two second target images are two or more consecutive images captured by the camera system. In some embodiments, one or more of the at least two second target images are a fused image generated by performing image fusion on two or more consecutive images captured by the camera system. Specifically, the processing device 120 may perform image fusion using an image fusion algorithm or a machine learning model. For example, the processing device 120 may perform image fusion using one of weighted average, pixel grayscale selection, PCA fusion, logical filtering, color space fusion, or the like, or any combination thereof. As another example, the processing device 120 may perform image fusion using a neural network model. In some embodiments, the processing device 120 may determine the motion change information of the target subject based on image change information of the fused image. The processing device 120 may determine image change information using the fused image, and then determine the motion change information (e.g., the motion status information) of the target subject based on the image change information. The image change information indicates a change of the positions of the ROI in the at least two second target images. During the operation process of the camera system, the quality of the image taken by the camera system may be affected by reasons such as illumination changes and environmental noise, which increases the detection uncertainty of the motion change information, and causes inaccurate detection of the motion change information. By performing image fusion on more than one consecutive image, useful information can be extracted from two or more images of the same scene and fused into a clearer image (e.g., the fused image), thereby expanding temporal and spatial information contained in the consecutive images, reducing interference from factors such as image noise and external illumination changes, and improving the detection accuracy of the motion change information.

In some embodiments, since metal objects worn by the target subject also affect a medical imaging scan (e.g., a CT scan), the processing device 120 may detect whether there are any unnoticed metal objects on the target subject based on at least one of the at least two second target images and/or the fused images. If there are some unnoticed metal objects on the target subject, the doctor or the target subject may be prompted to perform a secondary confirmation.

In some embodiments, the processing device 120 may determine image change information of the at least two second target images using a motion detection model. The at least two second target images may include the fused image. In response to determining that the at least two second target images include the ROI, the processing device 120 may image change information of the at least two second target images using the motion detection model. The processing device 120 may determine the motion change information of the ROI based on the image change information. The motion change information of the target subject may include motion change information of the ROI of the target subject. In some embodiments, the processing device 120 may use the motion change information of the ROI of the target subject as the motion change information of the target subject. By determining the motion change information of the ROI based on the motion detection model, a large amount of data can be processed quickly and accurately using the motion detection model, thereby improving the data processing efficiency and accuracy; by using the motion change information of the ROI as the motion change information of the target subject, invalid data can be prevented from being processed, thereby reducing the amount of data and improving the processing speed.

Merely by way of example only, as shown in FIG. 14A and FIG. 14B, since the field of view of the camera devices 1420 is not completely consistent with the scanning field of view of the medical imaging device, in order to better reflect the motion change of the ROI, the processing device 120 may detect whether the at least two second target images taken by the camera devices 1420 include the ROI through the motion detection model. In response to determining that the at least two second target images include the ROI, the processing device 120 may detect the image change information in the at least two second target images using the motion detection model, and then determine the motion change information of the ROI through the image change information.

In the previous embodiment, before detecting the ROI using the motion detection model, the processing device 120 may first fuse images of the same portion taken by the two camera devices 1420. For example, when the head is scanned, the processing device 120 may first obtain a clearer fused image of the head through image fusion. The processing device 120 may first determine whether the fused image includes the head and whether the head moves using the motion detection model. In response to determining that the fused image includes the head and the head moves, the processing device 120 may estimate image change information of the head, and then determine motion change information of the head through the image change information.

In some embodiments, the motion detection model may include various machine learning models. For example, the motion detection model may include a neural network model such as convolutional neural networks (CNN). An input of the motion detection model may include more than one second target image, and an output of the motion detection model may include image change information of the more than one second target image. The motion detection model may be obtained by training a preliminary model using a training set. The training set may include a body image data set and a label of the body image data set. The body image data set may include a plurality of images depicting a moving human body, and the plurality of images may include marker points of different body parts of the moving human body. The label of the body image data set may include image change information of the moving human body. Merely by way of example, assuming that the motion detection model is a CNN, during model training, a first module of the preliminary model may extract and learn deep features from the plurality of images of the body image data set through operations such as convolution and pooling, and a second module of the preliminary model may learn changes of different marker points based on the deep features, and when a preset training condition is met (e.g., a count of training iterations meets a preset number of times), obtain corresponding network model parameters to complete the training process and obtain a trained motion detection model. For the trained motion detection model, when more than one second target image is input into the motion detection model, the first module may identify the ROI and regional information (i.e., the deep features) of the ROI in the more than one second target image, and the second module may determine whether the ROI moves based on the regional information, and output the image change information of the ROI. By training the model with the body image data set, the motion detection model of which the performance and quality the meet the requirements can be obtained.

In some embodiments, the processing device 120 may transform the image change information into the motion status information using a mapping matrix of the camera system. The mapping matrix may be configured to transform two-dimensional coordinates of the at least two second target images into three-dimensional coordinates in space. In some embodiments, the mapping matrix may be a transformation matrix of coordinates between a scanning gantry coordinate system and an image coordinate system. The scanning gantry coordinate system is a 3D coordinate system established with the scanning gantry as a reference. The image coordinate system is a 2D coordinate system established with the second target image as references. The mapping matrix may include intrinsic and extrinsic parameters of the one or more camera devices. The intrinsic parameters are parameters related to the features of the one or more camera devices, including a focal length, an imaging origin, and a distortion coefficient (e.g., radial and tangential), etc. The extrinsic parameters are parameters that reflect a relative position of the camera device coordinate system and a world coordinate system, including a rotation vector, a translation vector, camera device coordinates, etc. The world coordinate system is a three-dimensional coordinate system established at any point in a three-dimensional space. For example, the world coordinate system may include the scanning gantry coordinate system, etc. The processing device 120 may transform the two-dimensional coordinates of the at least two second target images into the three-dimensional coordinates in space through the mapping matrix, thereby transforming the image change information into the motion status information in space. By using the mapping matrix, the image change information can be quickly transformed into the motion status information in space, thereby improving the transformation efficiency.

In some embodiments, the processing device 120 may obtain the mapping matrix between the camera device coordinate system and the scanning gantry coordinate system in various calibration manners (e.g., ZHANG, Zhengyou calibration method, etc.). Merely by way of example only, a reference object used during calibration may include a chessboard, a system mold, a steel ball mold, or any other mold containing a marker (e.g., a cross, a circle, a square, etc.).

In some embodiments, in response to determining that the motion status information of the target subject is larger than a motion threshold, the processing device 120 may determine that there is a motion of the ROI between the at least two second target images, and perform operation S1330 to perform artifact correction on the scanning data based on the motion status information. In response to determining that the motion status information of the target subject is not larger than the motion threshold, the processing device 120 may determine that the ROI does not move and repeat operations S1320 and S1330.

In S1340, a corrected image may be generated by performing, based on the motion change information, artifact correction on the scanning data. In some embodiments, the operation S1340 may be performed by the data correction module 260 shown in FIG. 2.

In some embodiments, the processing device 120 may correct the scanning data based on the motion status information and the motion time information. The corrected scanning data may be scanning data after motion compensation. The processing device 120 may generate the corrected scanning image by performing image reconstruction on the corrected scanning data. Since the motion change information includes both time information (i.e., the motion time information) and space information (i.e., the motion status information), and the motion change of the ROI of the target subject is also a status change in the time dimension and the space dimension, by correcting the scanning data based on the motion status information and the motion time information, the scanning data that needs to be corrected can be more accurately located, thereby improving the accuracy of image correction. Merely by way of example, assuming that the scanning data is view data (e.g., projection data), the processing device 120 may correct the view data in the corresponding motion time information of the target subject based on the motion status information, such that the raw data of CT imaging can be corrected from the time dimension and the space dimension, and the image reconstruction can be performed on the corrected scanning data, thereby improving the accuracy of medical imaging.

In some embodiments, the processing device 120 may obtain motion time information corresponding to at least one of the position change information and the orientation change information. The position change information and the orientation change information of the motion status information may be configured to reflect the position and orientation changes of the ROI. The processing device 120 may determine a portion of the scanning data corresponding to the motion time information, and compensate the at least a portion of the scanning data using at least one of the position change information and the orientation change information. It can be understood that during scanning, the movement of the ROI may occur at a portion of the scanning process, the scanning data acquired in the portion of the scanning process instead of the whole scanning process needs to be corrected. Merely by way of example, assuming that the scanning data is view data, the processing device 120 may first determine the corresponding motion time information according to at least one of the position change information and the orientation change information of the ROI. Then, the processing device 120 may determine the corresponding scanning data according to the motion time information, i.e., determine the view data within a time period from a start motion time to an end motion time in the motion time information. Finally, the processing device 120 may reversely compensate the view data according to at least one of the position change information and the orientation change information of the ROI. By determining and compensating the scanning data using the motion time information, invalid correction of the data (e.g., the scanning data acquired when the ROI of the patient does not move) can be avoided, thereby achieving accurate correction of the scanning data.

In some embodiments, the processing device 120 may generate the corrected scanning image based on the motion change information and the scanning data using a correction model. The processing device 120 may generate corrected scanning data by inputting the motion change information and the scanning data into a correction model. The processing device 120 may generate the corrected scanning image by performing image reconstruction on the corrected scanning data. For example, after the scanning, the processing device 120 may generate corrected scanning data by inputting the motion change information and all scanning data acquired in the whole scanning process into the correction model. The processing device 120 may generate the corrected scanning image by performing image reconstruction on the corrected scanning data. As another example, the processing device 120 may generate corrected scanning data by inputting the motion change information and scanning data acquired when the ROI moves (e.g., scanning data acquired corresponding to the motion time information) into the correction model. The processing device 120 may generate the corrected scanning image by performing image reconstruction on the corrected scanning data and original scanning data acquired when the ROI does not move. In this way, the artifact correction may be performed after scanning, or the artifact correction and the scanning process may be performed simultaneously.

The correction model may include various machine learning models. For example, the correction model may include a CNN model. An input of the correction model may include the motion change information and the scanning data, and an output of the correction model may include the corrected scanning data. By correcting the scanning data using the machine learning model, the data processing efficiency and quality can be improved. In some embodiments, the training mode of the correction model may be similar to that of the motion detection model, which is not repeated here.

For a training process of the correction model, training samples include motion change information of sample subjects and sample scanning data of the sample subjects. Labels of the training samples include scanning data of the sample subjects acquired without movement of the sample subjects. The sample scanning data is acquired when the sample subjects move.

In some embodiments, the motion detection model and the correction model may be obtained through joint training. A training set may include a body image data set and a label of the body image data set. The body image data set may include a plurality of images depicting a moving human body, and the plurality of images may include marker points of different body parts of the moving human body. The label of the body image data set may include image change information of the moving human body. The training set may further include sample scanning data of the moving human body acquired when the human body is moving, and scanning data of the human body acquired without movement of the human body. Merely by way of example, the processing device 120 may input the body image data set into a premilitary motion detection model to obtain output image change information of the ROI. The processing device 120 may transform the image change information into motion change information, and input the motion change information and the sample scanning data into a premilitary correction model to obtain output corrected scanning data. The processing device 120 may determine a first loss value based on the image change information of the body image data set and the output image change information, and determine a second loss value based on the output corrected scanning data and the scanning data of the human body acquired without movement of the human body. The processing device 120 may iteratively adjust parameters of the motion detection model based on the first loss value and the second loss value, and iteratively adjust parameters of the correction model based on the second loss value until a preset training condition is met (e.g., a count of training iterations meets a preset number of times), and the joint training of the model may be completed. Through the joint training of the motion detection model and the correction model, the quality of the final corrected image can be guaranteed, while saving the model training time and improving the model training efficiency.

In the embodiments of the present disclosure, the motion change information of the target subject is determined through the at least two images taken by the camera system, and image artifact correction is performed on the scanning image of the medical imaging device based on the motion change information, thereby reducing the motion artifacts in the image and improving the quality and accuracy of medical imaging. In some embodiments, during scanning, the processing device 120 may obtain, at preset intervals, at least two second target images. In some embodiments, during scanning, the processing device 120 may obtain at least two second target images in real time. For example, from the start of scanning, the camera system may capture real-time images of the target subject to monitor the target subject in real time. The processing device 120 may obtain every two adjacent images of the real-time images as two second target images. Alternatively, the processing device 120 may obtain every four adjacent images of the real-time images, generate a first fused image by fusing the first two images of the four adjacent images, generate a second fused image by fusing the last two images of the four adjacent images, and designate the first fused image and the second fused image as two second target images. The processing device 120 may determine motion change information of the target subject based on the at least two second target image according to operation S1330. In response to determining that the motion status information of the target subject is larger than a motion threshold, the processing device 120 may determine that there is a motion of the ROI between the at least two second target images. The processing device 120 may generate a corrected image by performing, based on the motion change information, artifact correction on the scanning data. In some embodiments, since the artifact correction is performed on the scanning data acquired when the ROI moves instead of all scanning data acquired during the whole scanning process, performing artifact correction does not affect the subsequent motion detection and scanning. During scanning, when the processing device 120 detects that the ROI moves, the processing device 120 may perform artifact correction on the scanning data acquired when the ROI moves while continuing detecting whether there is a motion of the ROI and controlling the medical imaging device to continue scanning the target subject. When the scanning is completed, the artifact correction is also completed. The processing device 120 may generate the corrected image by performing image reconstruction on the corrected scanning data. In this way, the efficiency of medical imaging can be improved.

In some embodiments, before scanning, the processing device 120 may perform a process to determine an operation status of the camera system. In response to determining that the camera system operates normally, the processing device 120 may perform the process 1300 to scan the target subject. In response to determining that the camera system is in a fault status, the processing device 120 may send a prompt information of the fault status to the user. A camera device with a normal status may replace the camera device in the fault status. The processing device 120 may perform the process 1300 to scan the target subject with the new camera device. In response to determining that the camera system is in an abnormal tendency status, the processing device 120 may send a prompt information of the abnormal tendency status to the user, and perform the process 1300 to scan the target subject. If the camera system has an abnormal tendency of deviation, the processing device 120 may determine an offset of the camera system using the second fault detection model. The processing device 120 may consider the offset of the camera system when transforming the image change information into the motion status information. More descriptions regarding determining the operation status of the camera system may be found in FIG. 11 and FIG. 12.

The operations of the illustrated processes 300, 400, 500, and 600 presented above are intended to be illustrative. In some embodiments, a process may be accomplished with one or more additional operations not described, and/or without one or more of the operations discussed. Additionally, the order in which the operations of a process described above is not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” may mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C #, VB. NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A method, implemented by a computing device including at least one processor and at least one storage device, the method comprising:
obtaining a first target image of a target subject, the first target image being taken by a camera system;
determining a marker in the first target image, the marker being configured to characterize a scanning position; and
determining a scanning range of a medical imaging device for scanning the target subject based on the marker.

2. The method of claim 1, wherein the marker includes at least one of an imaging position, a center line of the target subject, or a region of interest (ROI) of the target subject, the imaging position being configured to characterize a position on the target subject capable of being scanned by the medical imaging device.

3. The method of claim 2, wherein the determining a marker in the first target image includes:
determining the imaging position according to a scanning protocol and bed position information of a scanning bed.

4. The method of claim 2, wherein the determining a marker in the first target image includes:
identifying the target subject in the first target image; and
determining the center line of the target subject according to the identified target subject in the first target image.

5. The method of claim 2, wherein the determining a marker in the first target image includes:
identifying the target subject in the first target image;
acquiring scanning information of the target subject, the scanning information indicating the ROI of the target subject; and
marking, based on the scanning information and the identified target subject in the first target image, a contour of the ROI in the first target image.

6. The method of claim 5, wherein the marking, based on the scanning information and the identified target subject in the first target image, a contour of the ROI in the first target image includes:
marking the contour of the ROI in the first target image using a recognition model, an input of the recognition model including the first target image and the scanning information.

7. The method of claim 6, wherein the input of the recognition model further includes at least one of a medical history of the target subject or a historical medical image of the ROI of the target subject.

8. The method of claim 2, wherein the determining a scanning range of a medical imaging device for scanning the target subject based on the marker includes:
displaying the first target image including the marker; and
determining, based on the displayed marker, the scanning range of the target subject.

9. The method of claim 8, wherein:
the first target image includes real-time images of the target subject during a movement process of a scanning bed of the medical imaging device, and the real-time images reflects a real-time relative position relationship between the target subject and the imaging position during the movement process of the scanning bed; and
determining, based on the displayed marker, the scanning range of the target subject includes:
during the movement process of the scanning bed, determining the scanning range of the target subject according to the real-time relative position relationship.

10. The method of claim 9, wherein the during the movement process of the scanning bed, determining the scanning range of the target subject according to the real-time relative position relationship includes:
during the movement process of the scanning bed, receiving a confirmation instruction, the confirmation instruction including a confirmation instruction of a start position and a confirmation instruction of an end position; and
determining the scanning range of the target subject according to the start position and the end position, the start position being a first target position related to the scanning bed, the end position being a second target position related to the scanning bed.

11. The method of claim 8, wherein the determining, based on the displayed marker, the scanning range of the target subject includes:
determining at least one image position in the first target image by receiving a user instruction performed in the displayed image; and
determining the scanning range of the target subject based on the at least one image position.

12. The method of claim 2, wherein the determining a scanning range of a medical imaging device for scanning the target subject based on the marker includes:
automatically determining at least one image position in the first target image based on the ROI of the target subject; and
determining the scanning range of the target subject based on the at least one image position.

13. The method of claim 1, wherein the camera system includes at least one of a first camera device and a second camera device mounted inside a scanning cavity of the medical imaging device, the first camera device being disposed at a top of the scanning cavity, the second camera device being disposed on a side of the scanning cavity.

14. The method of claim 13, wherein the first target image of the target subject includes a first target image of a top view of the target subject taken by the first camera device.

15. The method of claim 13, wherein the first target image of the target subject includes a second image of a side view of the target subject taken by the second camera device.

16. The method claim 1, further comprising:
controlling a movement process of a scanning bed of the medical imaging device; and
in response to determining that the scanning range matches the imaging position, controlling the medical imaging device to scan the target subject.

17. The method of claim 16, further comprising:
acquiring scanning data of the scanning range of the target subject;
during acquiring the scanning data, obtaining at least two second target images of the target subject generated based on the camera system, the at least two second target images corresponding to different time points;
determining motion change information of the target subject according to the at least two second target images; and
generating a corrected scanning image by performing, based on the motion change information, artifact correction on the scanning data.

18. The method claim 1, further comprising:
obtaining a target depth image taken by the camera system and a historical depth image captured in the same scene as the target depth image by the camera system prior to the target depth image;
determining an operation status of the camera system according to at least one of the target depth image and the historical depth image, the operation status including at least one of a fault status, an abnormal tendency status, or a normal status; and
sending prompt information to a user based on the operation status of the camera system.

19. (canceled)

20. A method, implemented by a computing device including at least one processor and at least one storage device, comprising:
obtaining a target depth image taken by a camera system and a historical depth image captured in the same scene as the target depth image by the camera system prior to the target depth image, the camera system being mounted in a medical device operation room;
determining an operation status of the camera system according to at least one of the target depth image or the historical depth image, and the operation status including a fault status, an abnormal tendency status, or a normal status; and
sending prompt information to a user based on the operation status of the camera system.

21-34. (canceled)

35. A method, implemented by a computing device including at least one processor and at least one storage device, comprising:
obtaining scanning data of a target subject acquired by scanning the target subject by a medical imaging device;
during acquiring the scanning data, obtaining at least two second target images of the target subject generated based on a camera system, the at least two second target images corresponding to different time points;
determining motion change information of the target subject according to the at least two second target images; and
generating a corrected scanning image by performing, based on the motion change information, artifact correction on the scanning data.

36-54. (canceled)
